SEQUENCE LISTING (41 sequences)

SEQ ID NO: 1 - DNA, 22 bp, synthetic construct
  source 1..22: mol_type = other DNA
catccacaaccttcccgaacac

SEQ ID NO: 2 - DNA, 22 bp, synthetic construct
  source 1..22: mol_type = other DNA
acactgaaacacggaggcaaag

SEQ ID NO: 3 - DNA, 20 bp, synthetic construct
  source 1..20: mol_type = other DNA
tgtgtgtttgggaatctcca

SEQ ID NO: 4 - DNA, 20 bp, synthetic construct
  source 1..20: mol_type = other DNA
ctgataaaccaaccgggaca

SEQ ID NO: 5 - DNA, 25 bp, synthetic construct
  source 1..25: mol_type = other DNA
tgtgttattgttttcctggcatttc

SEQ ID NO: 6 - DNA, 20 bp, synthetic construct
  source 1..20: mol_type = other DNA
gcgacagcgtggatctacag

SEQ ID NO: 7 - DNA, 23 bp, synthetic construct
  source 1..23: mol_type = other DNA
ctgccagcacaagattcgtgagc

SEQ ID NO: 8 - DNA, 24 bp, synthetic construct
  source 1..24: mol_type = other DNA
gaccctgcttattctgatcggaaa

SEQ ID NO: 9 - DNA, 25 bp, synthetic construct
  source 1..25: mol_type = other DNA
aactggcaacggttctatcagctca

SEQ ID NO: 10 - DNA, 20 bp, synthetic construct
  source 1..20: mol_type = other DNA
ttctggtgaaagagcagcgg

SEQ ID NO: 11 - DNA, 20 bp, synthetic construct
  source 1..20: mol_type = other DNA
cagggcgatctggcgtctct

SEQ ID NO: 12 - DNA, 20 bp, synthetic construct
  source 1..20: mol_type = other DNA
ctgctgtcgatgaactcccg

SEQ ID NO: 13 - DNA, 21 bp, synthetic construct
  source 1..21: mol_type = other DNA
cttgggctaaagtagtcaccg

SEQ ID NO: 14 - DNA, 22 bp, synthetic construct
  source 1..22: mol_type = other DNA
gatggcaaggtgcagatacgtg

SEQ ID NO: 15 - DNA, 17 bp, synthetic construct
  source 1..17: mol_type = other DNA
gatgggcgggggaaaat

SEQ ID NO: 16 - DNA, 22 bp, synthetic construct
  source 1..22: mol_type = other DNA
cttgtactggcgtggattctgc

SEQ ID NO: 17 - DNA, 22 bp, synthetic construct
  source 1..22: mol_type = other DNA
gcgaaggagaagaccacgacat

SEQ ID NO: 18 - DNA, 22 bp, synthetic construct
  source 1..22: mol_type = other DNA
taggcttccctgcctctccaaa

SEQ ID NO: 19 - DNA, 23 bp, synthetic construct
  source 1..23: mol_type = other DNA
cattgcctggatgaagtttgacg

SEQ ID NO: 20 - DNA, 22 bp, synthetic construct
  source 1..22: mol_type = other DNA
gggttggtcttcactgaagtcc

SEQ ID NO: 21 - DNA, 20 bp, synthetic construct
  source 1..20: mol_type = other DNA
cgtggtggccatagacaaca

SEQ ID NO: 22 - DNA, 25 bp, synthetic construct
  source 1..25: mol_type = other DNA
cctctctcacagcatacatcagatg

SEQ ID NO: 23 - DNA, 23 bp, synthetic construct
  source 1..23: mol_type = other DNA
ctccgagactttcgaggaaatac

SEQ ID NO: 24 - DNA, 23 bp, synthetic construct
  source 1..23: mol_type = other DNA
gccattgaagttggtagccttca

SEQ ID NO: 25 - DNA, 22 bp, synthetic construct
  source 1..22: mol_type = other DNA
ccacagaccttccaggagaatg

SEQ ID NO: 26 - DNA, 23 bp, synthetic construct
  source 1..23: mol_type = other DNA
gtgcagttcagtgatcgtacagg

SEQ ID NO: 27 - DNA, 23 bp, synthetic construct
  source 1..23: mol_type = other DNA
gagagtgattgagagtggaccac

SEQ ID NO: 28 - DNA, 22 bp, synthetic construct
  source 1..22: mol_type = other DNA
cacaaccctctgcacccagttt

SEQ ID NO: 29 - DNA, 21 bp, synthetic construct
  source 1..21: mol_type = other DNA
gaaaagccatcgtcaaaaggg

SEQ ID NO: 30 - DNA, 21 bp, synthetic construct
  source 1..21: mol_type = other DNA
tggaagcagtaggtaaggaga

SEQ ID NO: 31 - DNA, 22 bp, synthetic construct
  source 1..22: mol_type = other DNA
gccactactgtgcctttgagtc

SEQ ID NO: 32 - DNA, 22 bp, synthetic construct
  source 1..22: mol_type = other DNA
ccctcagagaatcgccagtact

SEQ ID NO: 33 - DNA, 20 bp, synthetic construct
  source 1..20: mol_type = other DNA
cgctaaggcaactggaagac

SEQ ID NO: 34 - DNA, 20 bp, synthetic construct
  source 1..20: mol_type = other DNA
ccagaccactgggaaacact

SEQ ID NO: 35 - DNA, 25 bp, synthetic construct
  source 1..25: mol_type = other DNA
cattaaagatgccctgatgtatccc

SEQ ID NO: 36 - DNA, 20 bp, synthetic construct
  source 1..20: mol_type = other DNA
agtggtggtccgtggttgag

SEQ ID NO: 37 - DNA, 24 bp, synthetic construct
  source 1..24: mol_type = other DNA
atggtgcaaggctatcccaagagt

SEQ ID NO: 38 - DNA, 24 bp, synthetic construct
  source 1..24: mol_type = other DNA
gcctgttgttggagccaaactcaa

SEQ ID NO: 39 - DNA, 24 bp, synthetic construct
  source 1..24: mol_type = other DNA
tctgccagcacaagattcgtgagc

SEQ ID NO: 40 - DNA, 23 bp, synthetic construct
  source 1..23: mol_type = other DNA
ccttgatgccattaccagtctcc

SEQ ID NO: 41 - DNA, 22 bp, synthetic construct
  source 1..22: mol_type = other DNA
aaacagctccgcatcaacctgc

(19) United States
(12) Patent Application Publication
(10) Pub. No.: US 2026/0306525 A1
(43) Pub. Date: Oct. 8, 2026

(54) COMBINATION THERAPY GLUCOCORTICOIDS AND GINSENOSIDE

(71) Applicant: FYTAGORAS B.V., Leiden (NL)

(72) Inventors: Mahmoud HALIMA, Katwijk aan Zee (NL); Annemarie Henrietta MEIJER, Den Haag (NL); Mei WANG, Oegstgeest (NL); Marcel Johannes Maria SCHAAF, Voorhout (NL); Henricus Adriaan Anna Jacobus KORTHOUT, Heerhugowaard (NL); Albert VAN DUIJN, Oegstgeest (NL); Frederik Pius VAN HORSSEN, Brielle (NL)

(21) Appl. No.: 19/482,523

(22) PCT Filed: May 8, 2024
(86) PCT No.: PCT/NL2024/050234

§ 371 Date: Nov. 7, 2025

(30) Foreign Application Priority Data
May 8, 2023 (NL) 2034780

(51) Int. Cl. A61K 31/704; A61K 9/107; A61K 31/573; A61K 36/258; A61P 29/00

(52) U.S. Cl. CPC A61K 31/704; A61K 9/1075; A61K 31/573; A61K 36/258; A61P 29/00; A61K 2236/333; A61K 2236/53

(57) ABSTRACT

Combination therapy glucocorticoids and ginsenoside The invention relates to a ginsenoside formulation comprising a PPT-type ginsenoside at least partly incorporated in a micelle, preferably at least partly incorporated in an outer layer of the micelle, said micelle having a molecular weight of at least 10.000 Da and a ginsenoside for use in a method of prevention or treatment of a side-effect of glucocorticoid (GC) treatment, said method comprising administering a ginsenoside to a subject that is suffering from, or at risk of suffering from, one or more side-effect(s) of GC treatment. The invention also relates to a combination of a glucocorticoid (GC) and a ginsenoside for use in a method of prevention or treatment of an inflammatory disease, wherein said method comprises administering a GC and a PPT-type ginsenoside in a particular molar ratio.

The invention relates to a ginsenoside for use in the treatment of a side-effect of glucocorticoid (GC) treatment, a combination of a GC and a ginsenoside for use in the treatment of an inflammatory disease, a ginsenoside formulation and a pharmaceutical composition comprising a ginsenoside and a GC. The invention further relates to methods for preparing such a ginsenoside formulation and pharmaceutical composition and a product obtainable by a method for preparing a ginsenoside formulation.

GCs are widely used drugs for treating overactive inflammatory responses and chronic inflammatory diseases [1-3]. GCs such as dexamethasone, prednisolone, and beclomethasone are synthetic analogs of the steroid hormone cortisol, the primary GC hormone in our body which is secreted by the adrenal glands after stress. GCs can diffuse across the cell membrane and bind and activate an intracellular receptor, the glucocorticoid receptor (GR) [1-3]. Upon activation, GR dimers translocate to the nucleus where they function as transcription factors to regulate the transcription of a plethora of target genes. They modulate gene transcription through several mechanisms. First, they bind to DNA sequences known as glucocorticoid response elements (GREs) to alter the transcription of responsive genes [1-3]. This mode of action is referred to as transactivation as in most cases it leads to increased gene transcription. Second, GR can induce changes in gene transcription by interacting with other transcription factors such as NF-κB and AP-1, and thereby modulate their activity. Since this type of activity is suppressive in most cases, this mechanism is generally called transrepression [4-6].

GCs are highly effective immunosuppressive and anti-inflammatory drugs. They inhibit the production of pro-inflammatory cytokines and other effector molecules, alter monocyte recirculation, and induce lymphocyte death [5,6]. Classically, the anti-inflammatory function of GCs is based on the transrepression activity of GR, but in recent years it has become clear that transactivation of anti-inflammatory genes plays a role as well. The main issue that limits the clinical use of GCs, especially in patients requiring long-term treatment, is the severity of their side effects, which include diabetes, decreased cortisol levels, reduced growth velocity, wound healing disorders, tissue degeneration, osteoporosis, hypertension, weight gain and muscle weakness [5-11]. Most of the side effects of GCs are caused by the transactivation activity of GR [7-9], although GR's transrepression activity has been shown to contribute to decreased cortisol levels and osteoporosis.

Another problem accompanying chronic GC treatment is resistance to this therapy. Over time, the treatment may become less effective due to decreased GR activity and higher doses will often be required for longer periods to elicit anti-inflammatory activity, potentially worsening the side effects [12]. Although the exact mechanisms behind acquired GC resistance are yet to be fully understood, ligand-mediated downregulation (homologous downregulation) of GR is considered the hallmark of acquired GC resistance [12,13].

Therefore, there is an urgent need for developing more selective anti-inflammatory GC drugs, which maintain the anti-inflammatory activity, and do not cause severe side effects and the resistance to GC therapy.

The inventors realized that one or more of the above-mentioned drawbacks may be overcome by administering a ginsenoside to a subject that is suffering from or at risk of suffering from side-effects of GC treatment.

Accordingly, the invention relates to a ginsenoside for use in a method of prevention or treatment of a side-effect of GC treatment, said method comprising administering a ginsenoside to a subject that is suffering from, or at risk of suffering from, one or more side-effect(s) of GC treatment.

The inventors further realized that the side-effects of GC treatment may be effectively prevented or treated, whilst maintaining an anti-inflammatory effect, when a GC and a ginsenoside are administered in a specific molar ratio.

Accordingly, the invention further relates to a pharmaceutical composition comprising a GC, a ginsenoside and a pharmaceutically acceptable carrier, wherein the molar ratio between said GC and said ginsenoside is between about 1:1 and about 1:2000.

The inventors even further realized that solubility of a ginsenoside and optionally said GC may be significantly improved when incorporating said ginsenoside at least partly in a micelle. Improved solubility is particularly advantageous in case a relatively high concentration of a ginsenoside is desirable, for example to enable administration of a relatively high concentration of a ginsenoside to a subject in need thereof, or to facilitate the manufacturing of a pharmaceutical composition comprising a ginsenoside.

Accordingly, the invention further relates to a ginsenoside formulation comprising a PPT-type ginsenoside at least partly incorporated in a micelle, preferably at least partly incorporated in an outer layer of the micelle, said micelle having a molecular weight of at least 10.000 Da.

### FIGURES

FIG. 1. Rg1 induces additive anti-inflammatory effects to beclomethasone in the zebrafish tail fin wounding assay. (A) The chemical structures of the synthetic GC beclomethasone and the ginsenoside Rg1, glucose groups were indicated in Blue. (B) Schematic overview of the experimental design of the tail wounding assay. At 72 hpf, zebrafish larvae of the Tg(mpx:GFPⁱ¹¹⁴/mpeg1:mCherry^{umsF001}) line were treated with (combinations of) compounds for 6 h (2 h pre-wounding and 4 h post-wounding). At 74 hpf, tail fin wounding was performed at the indicated site (red line). At 78 hpf, the larvae were imaged by fluorescence microscopy, and the area for quantification of the fluorescently labeled neutrophils and macrophages (i.e., the area posterior to the tail vein) is indicated (red box). (C) The effects of vehicle (Veh) and different doses of beclomethasone (Bec), Rg1, and 10 μM beclomethasone in combination with different doses of Rg1 on the number of neutrophils that have migrated to the wounded site. The data shown (means±SEM) are pooled data from three independent experiments, each performed with 15 larvae per group. (D) Numbers of neutrophils and macrophages that have migrated towards the wounded site, after treatment with vehicle, 10 μM beclomethasone, 50 μM Rg1, or the combination of 10 μM beclomethasone with 50 μM Rg1. The data shown (means±SEM) are pooled from three independent experiments, each performed with 20 larvae per group. (E) Representative fluorescence microscopy images of wounding-induced migration of (GFP-labeled) neutrophils after treatment with vehicle, beclomethasone, dexamethasone, Rg1, or Rg1 in combination with beclomethasone. Scale bar: 100 μm. (F) The effect of beclomethasone, Rg1, and beclomethasone with Rg1 on the expression of il1b and il6. Expression analysis by qPCR was performed using total RNA from non-wounded larvae and wounded larvae, treated with either vehicle, 10 μM beclomethasone, 50 μM Rg1, or 10 UM beclomethasone with 50 μM Rg1. The relative expression levels in (F) were normalized to those of ppial in zebrafish. Data shown are means±SEM of three independent experiments (each performed in triplicate, with technical duplicates). Statistical significance in C, D and F is indicated: P<0.05 (*), 0.01 (**), and 0.001 (***) compared to the wounded vehicle group; P<0.05 (#), 0.01 (##), and 0.001 (###) for the combination treatment compared to beclomethasone.

FIG. S1. (A) Representative fluorescence microscopy images of wounding-induced migration of (GFP-labeled) neutrophils and (mCherry-labeled) macrophages after treatment with vehicle, beclomethasone (10 μM), Rg1 (50 μM), or Rg1 (50 μM) in combination with beclomethasone (10 μM). Scale bar: 100 μm. The white box indicates the counting area. (B) Representative fluorescence microscopy images of wounding-induced migration of neutrophils after treatment with dexamethasone (10 μM), or Rg1 (50 μM) in combination with dexamethasone (10 μM). Scale bar: 100 μm. (C) Numbers of neutrophils that have migrated towards the wounded site after treatment with vehicle, 10 μM dexamethasone, or the combination of 10 μM dexamethasone and 50 μM Rg1. The data shown (means±SEM) are pooled from three independent experiments, each performed with 15 larvae per group. Statistical significance in (C) is indicated: P<0.001 (***) compared to the Vehicle group; P<0.001 (###) for the combination treatment compared to dexamethasone.

FIG. 2. Rg1 binds with GR receptors and trigger an additive anti-inflammatory effects to beclomethasone in the Hela cell line. (A) In vitro determination of relative GR binding affinities using the PolarScreen Glucocorticoid Receptor (GR) Competitor Assay. Fluorescence polarization levels are plotted, reflecting the binding of a fluorescent ligand that is competed off the receptor by increasing concentrations of the compounds beclomethasone (Bec), dexamethasone (Dex), and Rg1. IC50 levels for each compound are indicated. Data shown are means±SEM of two individual experiments (performed in duplicate). (B) Nuclear translocation levels of GR in HeLa cells upon treatment with beclomethasone and Rg1, determined using immunocytochemistry and confocal microscopy, and the relative translocation levels (means±SEM of three individual experiments (performed in triplicate). (C) Representative confocal microscopy images of nuclear translocation levels of GR in HeLa cells upon treatment with beclomethasone and Rg1, showing GR (green) and DAPI staining (blue). (D) The effect of beclomethasone, Rg1, and beclomethasone with Rg1 on the expression of IL1B and IL8. Expression analysis was performed by qPCR using total RNA from HeLa cells treated without, or with TNF-α, and co-treated with vehicle, 0.01 μM beclomethasone, 20 μM Rg1, or 0.01 UM beclomethasone with 20 μM Rg1. The relative expression levels were normalized to those of 18S rRNA in HeLa cells and are shown on a log 2 scale. Data shown are means±SEM of two individual experiments (performed in duplicate) in A and of three independent experiments (each performed in triplicate, with technical duplicates) in B and D. Statistical significance in A, and D is indicated: P<0.05 (*), 0.01 (**), and 0.001 (***) compared to the wounded vehicle group; P<0.05 (#), 0.01 (##), and 0.001 (###) for the combination treatment compared to beclomethasone.

FIG. S2. Rg1 reduces both beclomethasone- and dexamethasone-induced side effects. (A) Representative images of the tail fins of zebrafish larvae at 5 dpf, wounded at 2 dpf and treated with Rg1 (50 μM) alone or in combination with beclomethasone (10 μM) or dexamethasone (10 μM). Scale bar: 100 μm. (B) Length of the regenerated tail fin at 5 dpf after tail fin amputation at 2 dpf, and treatment with vehicle (Veh), 10 μM dexamethasone (Dex), or 10 μM dexamethasone with 50 μM Rg1 from 0 to 5 dpf. (C) Relative EGFP intensities of Tg(9xGCRE-HSV.UI23:EGFP)^{ia20} reporter zebrafish larvae after exposure to the indicated treatments for 24 h at 2 dpf. The data shown in B and C (means±SEM) are pooled from three independent experiments, each performed with 15 larvae per group. Statistical significance in (C) is indicated: P<0.001 (***) compared to the vehicle group; P<0.001 (###) for the combination treatment compared to dexamethasone.

FIG. 3. Rg1 co-treatment strongly reduces beclomethasone-induced side effects. (A) Length of the regenerated tail fin at 5 dpf after tail fin wounding at 2 dpf, and treatment with vehicle (Veh), 10 μM beclomethasone (Bec), 50 μM Rg1, or 10 μM beclomethasone with 50 μM Rg1 from 0 to 5 dpf. The data shown (means±SEM) are pooled from three independent experiments, each performed with 15 larvae per group. (B) Length of larvae at 5 dpf, after 5-day treatment with vehicle, beclomethasone, Rg1, or beclomethasone with Rg1. The data shown (means±SEM) are pooled from three independent experiments, each performed with 15 larvae per group. (C, D) Whole body glucose levels, determined using a colorimetric assay on samples from 5 dpf larvae treated from 0 to 4 dpf with vehicle, 10 μM beclomethasone, 50 μM Rg1, or 10 μM beclomethasone with 50 μM Rg1 from 0 to 5 dpf (in C), and with different beclomethasone doses (0.1, 1, 5, 10 and 15 μM) alone and in combination with 50 μM Rg1 (in D). Data shown are means±SEM of three independent experiments (each performed in triplicate, with technical duplicates). (E, F) Whole body cortisol levels, determined using ELISA on samples from 5 dpf larvae treated from 0 to 4 dpf with vehicle, 10 μM beclomethasone, 50 μM Rg1, or 10 μM beclomethasone with 50 μM Rg1 (in E), and with different beclomethasone doses (0.1, 1, 5, 10 and 15 μM) alone and in combination with 50 μM Rg1 (in F). (G) Relative EGFP intensities of Tg(9xGCRE-HSV.UI23:EGFP)^{ia20} reporter zebrafish larvae (3 dpf) after exposure to the indicated treatments for 24 h (starting at 2 dpf). The data shown (means±SEM) are pooled from three independent experiments, each performed with 15 larvae per group. (H) Representative fluorescence microscopy images of Tg(9xGCRE-HSV.UI23:EGFP)^{ia20} reporter zebrafish larvae (3 dpf) after exposure to the indicated treatments for 24 h (starting at 2 dpf). Scale bar: 100 μm. (I) The fkbp5 and pck1 mRNA levels, studied by qPCR using total RNA from 3 dpf larvae without wounding or with wounding after treatment with vehicle, beclomethasone, Rg1, or beclomethasone with Rg1. The treatment lasted 6 h (2 h pre- and 4 h post-wounding). The relative expression values were normalized to those of ppial and shown on a log 2 scale. The data shown are means±SEM of three independent experiments, each performed in triplicate with technical duplicates. In graphs A, B, C, E, G, and I, statistical significance is indicated: P<0.05 (*), 0.01 (**), and 0.001 (***) compared to the vehicle group, and wounded vehicle group in (I); P<0.05 (#), 0.01 (##), and 0.001 (###) for the combination treatment compared to beclomethasone. In graphs D, F statistical significance indicated: P<0.05 (*), 0.01 (**), and 0.001 (***) compared to the Rg1 group.

FIG. 4. GC sensitivity is not reduced after long-term beclomethasone and Rg1 co-treatment. The mRNA levels of fkbp5 (A), pck1 (B), nfkbiaa (C), and gr (D) were studied by qPCR using total RNA from 5 dpf larvae without wounding, after short- or long-term treatment with vehicle, beclomethasone (10 μM), Rg1 (50 μM), or beclomethasone (10 μM) with Rg1 (50 μM). The short-term treatment lasted 6 h. In the long-term treatment, larvae were treated for 5 days (from 0 to 5 dpf). The relative expression values were normalized to those of ppial and shown on a log 2 scale. The data shown are means±SEM of three independent experiments, each performed in triplicate with technical duplicates. Statistical significance is indicated: P<0.001 (***) compared to the vehicle group; P<0.001 (###) for the combination treatment compared to treatment with beclomethasone alone; P<0.05 (+), 0.01 (++), and 0.001 (+++) for the long-term treatment compared to the corresponding short-term treatment.

FIG. S4. Rg1 restores the GC sensitivity in Hela cells that is reduced by long-term high-dose GC treatment. (A, B) The effects of Rg1, beclomethasone as an individual treatment or in combination on the relative IL1B mRNA level (determined by qPCR) at different time points after TNF-α treatment during short-term treatment (in A) or long-term treatment (in B). The, control group reflects a vehicle-treated group in the absence of TNF-α. (C, D) The effects of the combination treatment of Rg1 with dexamethasone on the expression of IL1B (in C) and NFKBIA (in D) after short- or long-term treatment. In A-D, the short-term treatment consisted of 6 h of compound co-treatment with TNF-α, and in the long-term treatment, cells were treated with the compounds for 30 h, consisting of 24 h plus 6 h of compound co-treatment with TNF-α. The relative expression values were normalized to those of 18S rRNA and shown on a log 2 scale. The data shown are means±SEM of three independent experiments, performed in triplicate with technical duplicates. Statistical significance is indicated: P<0.05 (*), 0.01 (**), and 0.001 (***) compared to the corresponding vehicle group; P<0.05 (#), 0.01 (##), and 0.001 (###) for the combination treatment compared to beclomethasone or dexamethasone alone; P<0.05 (+), 0.01 (++), and 0.001 (+++) for the long-term treatment compared to the corresponding short-term treatment.

FIG. 5. Rg1 restores the GC sensitivity in Hela cells that is reduced by long-term high-dose GC treatment. (A-G) Relative mRNA levels in HeLa cells for IL1B (A), MMP9 (B), IL8 (C), FKBP5 (D), NFKBIA (E), GILZ (F), SGK1 (G), determined by qPCR. In A, HeLa cells were treated (short- or long-term) with increasing doses of beclomethasone (0.01, 0.1 and 1 μM), Rg1 (20, 100 and 500 μM) or co-treatment of Rg1 (20, 100 and 500 μM) with beclomethasone (0.01 μM). In B-G, HeLa cells were treated with vehicle, beclomethasone (1 μM), Rg1 (20 μM), or beclomethasone with Rg1, in the absence and presence of TNF-α. The short-term treatment consisted of 6 h of compound co-treatment with TNF-α, and in the long-term treatment, cells were treated with the compounds for 30 h, consisting of 24 h plus 6 h of compound co-treatment with TNF-α. The relative expression values were normalized to those of 18S rRNA and shown on a log 2 scale. The data shown in (A-G) are means±SEM of three independent experiments, each performed in triplicate with technical duplicates. Statistical significance is indicated: P<0.05 (*), 0.01 (**), and 0.001 (***) compared to the corresponding vehicle group; P<0.05 (#), 0.01 (##), and 0.001 (###) for the combination treatment compared to beclomethasone; P<0.05 (+), 0.01 (++), and 0.001 (+++) for the long-term treatment compared to the corresponding short-term treatment.

FIG. S5. Representative gels of western blots showing that Rg1 inhibits the effect of beclomethasone-induced downregulation of GR in HeLa cells. (A) Western blots for GR (and β-actin) were obtained using (duplicate) protein samples from Hela cells after short-term treatment (6 h) with the indicated compounds. (B) Western blots for GR (and β-actin) were obtained using (duplicate) protein samples from HeLa cells after long-term treatment (24 h) with the indicated compounds. (C) Western blots for GR (and β-actin) were obtained using protein samples from HeLa cells after long-term treatment (24 h) with the indicated compounds, in the absence and presence of cycloheximide (5 μg/ml). ImageJ was used to determine the integrated intensity of the GR and β-actin protein bands.

FIG. 6. Rg1 inhibits the beclomethasone-induced downregulation of GR in Hela cells. (A) The effects of beclomethasone (1 μM) and/or Rg1 (20 μM) treatment on GR mRNA levels, determined by qPCR, after short- and long-term treatment, in the absence and presence of actinomycin-D (1 ng/ml). The relative expression values were normalized to those of 18S rRNA. Data shown are means±SEM of three independent experiments (each performed in triplicate with technical duplicates) and are plotted on a log 2 scale. (B) The effects of beclomethasone and/or Rg1 treatment on GR protein levels, determined using western blotting, after short- and long-term treatment (6 and 24 h respectively). (C) The effects of beclomethasone and/or Rg1 treatment on GR protein levels in the absence and presence of cycloheximide (5 μg/ml), after long-term treatment. Data shown have been normalized to the level of β-actin and are means±SEM of four (in A) independent experiments, each performed in duplicate, or six (in B) independent experiments, each performed as a single measurement. Statistical significance is indicated: P<0.05 (*), 0.01 (**), and 0.001 (***) compared to the corresponding vehicle group; P<0.05 (#), 0.01 (##), and 0.001 (###) for the combination treatment compared to beclomethasone alone; P<0.05 (+), 0.01 (++), and 0.001 (+++) for the long-term treatment compared to the corresponding short-term treatment.

FIG. 7. The anti-inflammatory action of monoglycosylated ginsenosides requires Gr function, but not glucosylceramidase beta 2 (Gba2). A. Structures of the compounds used for treatment: Beclomethasone (Bec), Protopanaxatriol (PPT), F1, Rh1, and Rg1. Glucose (Glc) groups conjugated to the steroid backbone are indicated in blue. B. Schematic overview of the experimental approach. Zebrafish larvae from the Tg(mpx: GFPi114/mpeg1:mCherry-FumsF001) line were subjected to tail wounding at 74 hpf. Chemical compound treatments started at 2 h before wounding and were continued for 4 h after wounding. At this time point, the number of neutrophils that had migrated to the wounded area (indicated by the red box) was determined. C. The number of migrated neutrophils and macrophages upon compound treatment at 4 hours after wounding. D. The number of migrated neutrophils upon compound treatment in the absence and presence of the Gba2-inhibitor MZ31 at 4 hours after wounding. E. Relative mRNA levels for il1b, il6, i18, mmp9, and mmp13, determined by qPCR, before and after wounding and upon compound treatment. Data shown are means±SEM and represent the average of data pooled from three individual experiments (n=60) in C and D, or the average of three individual experiments (performed in triplicate) in E. Statistical significance was determined by one- or two-way ANOVA and Tukey's post hoc test. Significant difference from the wounded Veh groups (in C, E), or from the corresponding Veh group (Veh/Veh or Veh/MZ31; in D) is indicated by ***(P<0.001). The difference from the Veh group treated with the same compound (hatched versus non-Hatched bars) is indicated by ###(P<0.001). Significant difference from the PPT group (in E) is indicated by $$$ (P<0.001) and the difference from the F1 group (in E) is indicated by &&& (P<0.001).

FIG. S7. Fish Embryo Acute Toxicity Test (FET) for studied ginsenosides and beclomethasone (A, B). Embryos were exposed to different concentrations of those compounds to assess their toxicity. A negative control (nC), solvent control (sC), and positive control (pC) group were included in the experiment. A. Hatching rate (%) at 48 and 72 hpf. B. Survival rate (%) at 96 and 120 hpf. C. The number of migrated neutrophils upon compound treatment in wild type (gr^{+/+}) and Gr deficient (gr^{-/-}) individuals upon treatment with Bec, PPT, F1, Rh1, or Rg1. Data shown are means±SEM and represent the average of data pooled from two individual experiments (n=100) in A and B, and from three individual experiments (n=60) in C. Statistical significance was determined by one- or two-way ANOVA and Tukey's post hoc test. A significant difference from the Veh (NC) group (same time point) in (A, B), or from the corresponding Veh group (gr^{+/+} or gr^{-/-}; C) is indicated by ***(P<0.001) and the difference from the gr^{+/+} group treated with the same compound (hatched versus non-hatched bars; C) is indicated by ###(P<0.001).

FIG. 8. Ginsenosides show a strong reduction in side effects compared to beclomethasone. A. Whole-body glucose levels, determined by colorimetric assay, in 5 dpf zebrafish larvae treated (from 2 hpf until 5 dpf) with beclomethasone or ginsenosides PPT, F1, Rh1 or Rg1 from 2 hpf. B. Whole-body glucose levels in 5 dpf larvae, upon wounding at 2 dpf, and compound treatment with or without MZ31 from 2 hpf until 5 dpf. C. Whole-body cortisol levels, determined by ELISA, in 5 dpf zebrafish larvae, treated with beclomethasone or ginsenosides PPD, F2, or Rb1 (from 2 hpf until 4 dpf). D. Whole-body cortisol levels in 5 dpf larvae, upon wounding at 2 dpf, and compound treatment with or without MZ31 (from 2 hpf until 4dpf). E. Body length of zebrafish larvae at 5 dpf upon compound treatment from 2 hpf until 5 dpf. F. Length of the regenerated tail fin of 5 dpf larvae, wounded at 2 dpf, upon compound treatment with or without MZ31 (from 2 hpf until 5 dpf). G. Representative fluorescence microscopy images of 3 dpf embryos from experimental groups presented in H. Scale bar: 200 μm. H. Relative GFP fluorescence levels after 24 h compound treatment in 3 dpf embryos from the Tg(9x GCRE-HSV.UI23:EGFP) line, which is a reporter line for the transactivation activity of Gr. I. Relative mRNA levels for fkbp5, determined by qPCR, before and after wounding and upon compound treatment. Data shown in A, B, C, D and I are means±SEM and averages of three individual experiments (performed in triplicate). Data shown in E, F, and H are means±SEM and represent the average of data pooled from three individual experiments (n=60). Statistical significance was determined by one- or two-way ANOVA and Tukey's post hoc test. Significant difference from the Veh group (in A, C, E, H) or the corresponding Veh group (Veh/Veh or Veh/MZ31; in B, D, F) is indicated by *(P<0.05), **(P<0.01), *(P<0.001), the difference from the group treated with the same compound (hatched versus non-hatched bars; in B, D, F) is indicated by #(P<0.05), ##(P<0.01), ###(P<0.001)

FIG. S8A. Representative brightfield microscopy images of tail fins of 5 dpf larvae from experimental groups presented in FIG. 2F. Arrowheads indicate the site of amputation and any tissue observed to right of this point is regenerated. Scale bar: 100 μm. B. Relative mRNA levels for pck1 and nfkbiaa determined by qPCR, before and after wounding and upon compound treatment. Data shown are means±SEM and averages of three individual experiments (performed in triplicate). Statistical significance was determined by two-way ANOVA and Tukey's post hoc test. Significant difference from the Veh group is indicated by ***(P<0.001).

FIG. 9. Anti-inflammatory effects and putative side effects of ginsenoside Re on zebrafish after wound induction by tail amputation. A: effect of Re and GC (prednisone) on the number on neutrophils at the amputation site; B: effect of Re and GC (prednisone) on the number of macrophages at the amputation site; C: effect of Re and GC (prednisone) on whole body glucose level; D: effect of Re and GC (prednisone) on whole body cortisol and E: effect of Re and GC (prednisone) on regenerative tissue. Veh: vehicle.

FIG. 10A. Appearance of solutions of ginsenoside in water after micelle formation. From left to right: 10 mg/ml pure Re solved in ethanol prior to the micelle formatting procedure; 10 mg/mL pure Re solved in H₂O after the procedure of micelle formation; 150 mg/mL Re/Rg1 enriched ginseng extract in ethanol obtained by purification and decolorization on a D101 and D941 resin respectively; 150 mg/mL Re/Rg1 enriched ginseng extract after micelle formation. B: microscopic view (400×) of crystals of Re formed by solving the Re film in H₂O during the procedure of micelle formation

FIG. 11: Size exclusion chromatography to determine the molecular weight and composition of the micelles. 1 mL of an aqueous solution containing Re/Rg1 enriched ginseng extract and dexamethasone was loaded on a 17 mL Sephadex G50 size exclusion chromatography column after micelle formation and eluted with H₂O at a rate of 0.4 mL/min. Fractions of 2 mL were collected. The column was calibrated using blue dextran (MW 2.000.000 Da) and erythrosine (MW 880 Da) as molecular weight markers (see arrows for peak elution of the markers). The presence of and concentration of ginsenosides Re, Rg1 and dexamethasone was determined and calculated by means of HPLC.

FIG. 12: HPLC chromatograms of fractions relevant for or obtained during size exclusion chromatography. A: HPLC profile and peak location of pure dexamethasone at 203 nm (upper panel) and 241 nm (lower panel); B: 203 nm HPLC chromatogram of fraction 15 eluted at 30 mL showing the presence of Rg1 and Re as the major peaks; C: 241 nm HPLC chromatogram of fraction 15 eluted at 30 mL showing the presence of dexamethasone.

FIG. 13: % inhibition of 10 μM beclomethasone, 80 μM Rg1 and a combination of 10 μM beclomethasone and 80 μM Rg1 on migration of neutrophils to the wounded site.

### SUMMARY OF THE INVENTION

The invention relates to a ginsenoside formulation comprising a PPT-type ginsenoside at least partly incorporated in a micelle, preferably at least partly incorporated in an outer layer of the micelle, said micelle having a molecular weight of at least 10.000 Da, preferably a molecular weight of at most 100.000 Da.

In a preferred aspect, said ginsenoside is a PPT-type ginsenoside, preferably selected from Rg1, Re and a combination thereof.

The invention further relates to a method for preparing a ginsenoside formulation according to the invention, said method comprising
(a) providing a ginsenoside extract from a plant or plant part of the genus Panax, preferably Panax ginseng;
(b) contacting said ginsenoside extract with an aqueous solvent mixture, preferably wherein said aqueous solvent mixture comprises a mixture of water and a non-aqueous solvent, preferably an alcoholic solvent, more preferably selected from methanol, ethanol, propanol and isopropanol, to obtain a ginsenoside mixture;
(c) at least partly removing said aqueous solvent mixture from said ginsenoside mixture; and optionally
(d) reconstituting the ginsenoside mixture obtained in step c) in water to obtain a ginsenoside formulation.

In preferred methods, the method comprises subjecting said ginsenoside extract to a step of column chromatography using a non-polar resin as a stationary phase, preferably a D101 macroporous resin and/or subjecting said ginsenoside extract to a step of decolorization, preferably using a D941 macroporous resin.

The invention further relates to a ginsenoside formulation obtainable by a method according to the invention, preferably comprising a PPT-type ginsenoside at least partly incorporated in a micelle, preferably at least partly incorporated in an outer layer of the micelle, said micelle having a molecular weight of at least 10.000 Da and/or a molecular weight of at most 100.000 Da.

In a preferred ginsenoside formulation obtainable by a method according to the invention, said ginsenoside is a PPT-type ginsenoside, preferably selected from Rg1, Re and a combination thereof.

Further, the invention relates to a ginsenoside for use in a method of prevention or treatment of a side-effect of GC treatment, said method comprising administering a ginsenoside to a subject that is suffering from, or at risk of suffering from, one or more side-effect(s) of GC treatment.

Preferably, said side-effect is selected from GC-induced diabetes, GC-induced decreased cortisol levels, GC-induced reduced growth velocity, GC-induced wound healing disorders, GC-induced tissue degeneration, GC-induced osteoporosis, GC-induced hypertension, GC-induced weight gain and GC-induced muscle weakness.

The invention further relates to ginsenoside formulations according to the invention for use according to the invention.

The invention further relates to a combination of a GC, preferably selected from the group of beclomethasone and dexamethasone, and a ginsenoside for use in a method of prevention or treatment of an inflammatory disease, wherein said method comprises administering a GC and a ginsenoside in a molar ratio of between about 1:1 and about 1:2000 to a subject in need thereof, wherein said molar ratio is between about 1:4 and about 1:1000, more preferably between about 1:8 and about 1:20.

In preferred embodiments, the invention relates to the combination for use according to the invention, wherein said GC is administered in a therapeutically ineffective amount, preferably at a dose of at most 0.07 mg/kg/day, preferably at most 0.007 mg/kg/day and/or wherein said ginsenoside is administered in a therapeutic ineffective amount, preferably at a dose of at most 5.5 mg/kg/day, more preferably at most 0.05 mg/kg/day.

In preferred embodiments, the invention relates to the combination for use according to the invention, wherein said inflammatory disease is selected from asthma, allergic rhinitis, hay fever, urticaria, atopic eczema, chronic obstructive pulmonary disease, inflammation of the joints, muscles and tendons, lupus, inflammatory bowel disease, such as Crohn's disease and ulcerative colitis, giant cell arteritis and polymyalgia rheumatica, and multiple sclerosis.

Preferably, the invention relates to combinations for use according to any one of claims 8 to 13, wherein said ginsenoside is at least partly incorporated into a micelle, preferably at least partly incorporated in an outer layer of a micelle, said micelle having a molecular weight of at least 10.000 Da and/or a molecular weight of at most 100.000 Da. Preferably, said ginsenoside is a PPT-type ginsenoside, preferably selected from Rg1, Re and a combination thereof.

The invention further relates to a pharmaceutical composition comprising a GC, a ginsenoside, preferably wherein the ginsenoside is a protopanaxatriol (PPT)-type ginsenoside, more preferably selected from Rg1 and Re and a combination thereof and a pharmaceutically acceptable carrier, wherein the molar ratio between said GC and said ginsenoside is between about 1:1 and about 1:2000, preferably between about 1:4 and about 1:1000, more preferably between 1:8 and 1:20.

Preferably, in the pharmaceutical composition according to the invention, said ginsenoside is at least partly incorporated into a micelle, preferably at least partly incorporated in an outer layer of a micelle, said micelle having a molecular weight of at least 10.000 Da and/or at most 100.000 Da.

In an aspect, the invention relates to a pharmaceutical composition according to the invention, wherein said pharmaceutical composition is formulated as a cream, a lotion, a balm, a hydrogel, an ointment, a foam, a gel, a spray, a tablet, a capsule, a lozenge, a topical solution, a topical suspension, an aerosol, an injectable and a syrup. Most preferably, the pharmaceutical composition is formulated as a hydrogel.

The invention further relates to the pharmaceutical composition for use in a method of prevention or treatment of an inflammatory disease, preferably wherein said inflammatory disease is selected from asthma, allergic rhinitis, hay fever, urticaria, atopic eczema, chronic obstructive pulmonary disease, inflammation of the joints, muscles and tendons, lupus, inflammatory bowel disease, such as Crohn's disease and ulcerative colitis, giant cell arteritis and polymyalgia rheumatica, and multiple sclerosis.

### DETAILED DESCRIPTION

The term “or” as used herein is defined as “and/or” unless specified otherwise.

The term “a” or “an” as used herein is defined as “at least one” unless specified otherwise.

The term “substantial(ly)” or “essential(ly)” is generally used herein to indicate that it has the general character or function of that which is specified. When referring to a quantifiable feature, these terms are in particular used to indicate that it is for at least 75%, more in particular at least 90%, even more in particular at least 95% of the indicated feature.

In the context of the present application, the term “about” means generally a deviation of 15% or less from the given value, in particular a deviation of 10% or less, more in particular a deviation of 5% or less.

When referring to a noun in the singular, the plural is meant to be included, unless it follows from the context that it should refer to the singular only.

“Ginsenosides” are a class of steroid glycosides and triterpene saponins found in the plant genus Panax. Ginsenosides are commonly referred to by the name “Rx”, wherein the x describes the chromatographic polarity in alphabetical order, although there are also ginsenosides described using a different name, such as ginsenoside F1. Thus, Ra is one of the least polar ginsenoside described thus far and Rb is more polar than Ra.

Ginsenosides are characterized by a dammarane scaffold—also known under the systemic name (1R,3aR,3bR,5aS,9aS,9bR,11aR)-1-[(2R)-6-Methylheptan-2-yl]-3a,3b,6,6,9a-pentamethylhexadecahydro-1H-cyclopenta[α]phenanthrene—typically with a sugar moiety attached thereto, although ginsenosides lacking a sugar moiety have also been described, such as ginsenoside “aglycon-PPT”. Ginsenosides are usually divided into two major subclasses, i.e. 20(S)-protopanaxadiol (PPD) ginsenosides (formula I) and 20(S)-protopanaxatriol (PPT) ginsenosides (formula II). PPD and PPT-type ginsenosides are distinguished from one another in the position of the sugar moiety to the dammarane scaffold. In PPT-type ginsenosides, the sugar moiety is attached to the 6-position of the scaffold, whereas in PPD-type ginsenosides the sugar moiety is attached to the 3-position of the scaffold.

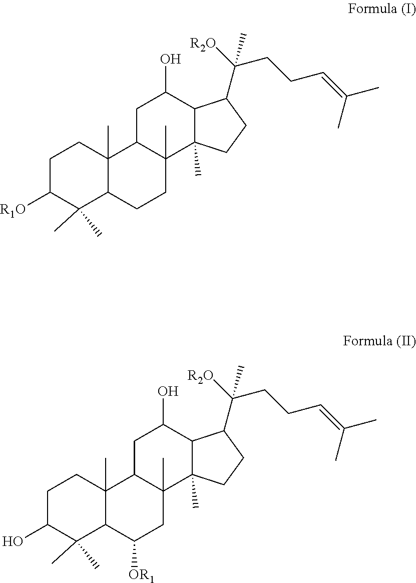

Examples of protopanaxadiol-type ginsenosides include Rb1, Rb2, Rb3, Rc, Rd, Rg3, Rh2 and Rs1. Examples of protopanaxatriol-type ginsenosides include Re, Rf, Rg1, Rg2 and Rh1. Further, some rare ginsenosides are described, such as the ocotillol saponin F11 (24-R-pseudoginsenoside) and the pentacyclic oleanane saponin Ro (3,28-O-bisdesmoside).

“Glucocorticoids” (GC) refer herein to a class of steroid hormones that have an ability to bind to the glucocorticoid receptor. GCs are commonly prescribed in the treatment of (chronic) inflammatory diseases, including eczema, asthma and rheumatoid arthritis. Cortisol, also referred to as hydrocortisone, is an endogenous GC. Synthetic analogues of cortisol are also described, which typically have one or more changed properties compared to cortisol. Examples of synthetic GCs include dexamethasone, betamethasone, prednisolone, methylprednisolone, triamcinolone, deflazacort, fludrocortisone acetate, fludrocortisone acetate, aldosterone, beclomethasone, hydrocortisone or a prodrug thereof.

With the term prodrug as used herein is meant an analogue of a GC that is converted to a biologically active GC in vivo. Examples of prodrugs of GCs include cortisone and prednisone.

The term “micelle” refers to a substantially spherical particle, comprising an outer layer comprising one or more amphiphilic molecules and an inner cavity. The outer layer typically comprises a hydrophilic outer surface and a hydrophobic inner centre. Typically, in an aqueous solution, the hydrophilic part of the micelle is in contact with an aqueous medium, whereas the hydrophobic part of the micelle is shielded from the aqueous medium. Said inner cavity is usually formed by the outer layer, and may encompass one or more small molecules, such as a therapeutic molecule.

In the context of the present application, the hydrophilic part of the micelle is usually formed by the hydrophilic part of the ginsenosides, typically the sugar moiety, whereas the hydrophobic part of the micelle is usually formed by the hydrophobic part of the ginsenoside, typically the dammarane moiety of the ginsenoside.

With the term “therapeutically effective amount” as used herein, is meant an amount of an active principle, e.g. a GC or a ginsenoside, when administered during a period of time necessary, that is effective to achieve an anti-inflammatory effect in a subject in need thereof. With the term “therapeutically ineffective amount” is meant any amount of an active principle, such as a GC or a ginsenoside, that is lower than a therapeutic effective amount, and which essentially does not exhibit a significant anti-inflammatory effect in a subject in need thereof, when administered for a sufficient period of time and in absence of another anti-inflammatory compound.

### Ginsenoside for Use in the Treatment of a Side-Effect of GC Treatment

Although GCs are effective anti-inflammatory drugs, their long-term use is associated with a number of drawbacks.

For example, long-term use of GC's is associated with decreased glucocorticoid receptor (GR) activity and thus increased GC resistance. Consequently, in order to maintain effectiveness of GCs, the dosage is typically increased.

However, in particular long-term use of GCs is associated with the occurrence of severe side-effects, including diabetes, decreased cortisol levels, reduced growth velocity, wound healing disorders, tissue degeneration, osteoporosis, hypertension, weight gain and muscle weakness.

The occurrence and severity of these side-effects is further usually enhanced at increased dosage, which is often required to overcome the decreased glucocorticoid receptor activity and maintain efficacy.

The inventors surprisingly realized that a ginsenoside can effectively prevent or alleviate the side-effects of GC treatment and can furthermore prevent GC resistance. This would significantly increase the therapeutic options for the treatment of anti-inflammatory diseases.

Without wishing to be bound by any theory, the inventors believe that a ginsenoside, in particular Rg1, acts as a competitive antagonist of the glucocorticoid receptor (see e.g. Examples and FIGS. 3D, F). In more detail, the inventors envisage that selective binding of a ginsenoside to the glucocorticoid receptor induces transrepression but not transactivation, the latter being linked to the occurrence of side-effects.

Accordingly, in an aspect the invention relates to a ginsenoside for use in a method of prevention or treatment of a side-effect of GC treatment, said method comprising administering a ginsenoside to a subject that is suffering from, or at risk of suffering from, one or more side-effect(s) of GC treatment.

### Ginsenoside

The ginsenoside for use according to the invention may in principle be any ginsenoside that exerts an anti-inflammatory effect and that is capable of mediating the side-effects of GC treatment.

Said ginsenoside is preferably a 20(S)-protopanaxadiol (PPD)-type ginsenoside or a 20(S)-protopaxanatriol (PPT)-type ginsenoside, more preferably a PPT-type ginsenoside.

Preferably, said ginsenoside is selected from Rg1, Re, Rf, Rg2, F1, aglycon-PPT and Rh1 or a combination thereof, more preferably said at least one ginsenoside is selected from Rg1 and Re or a combination thereof.

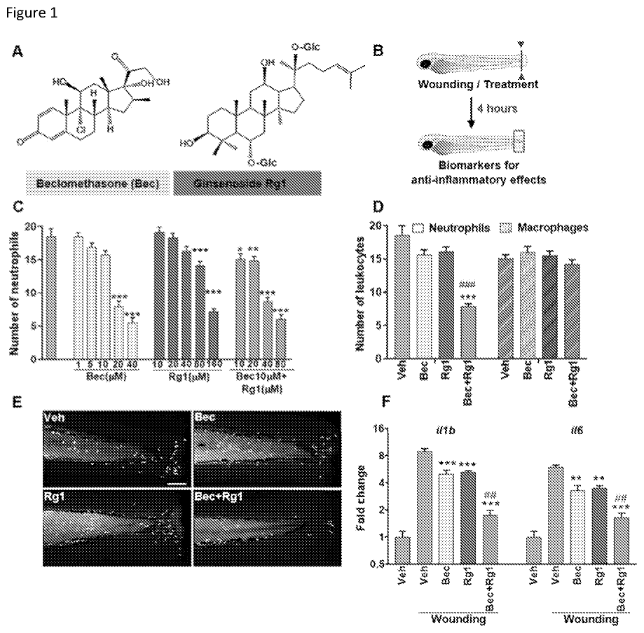

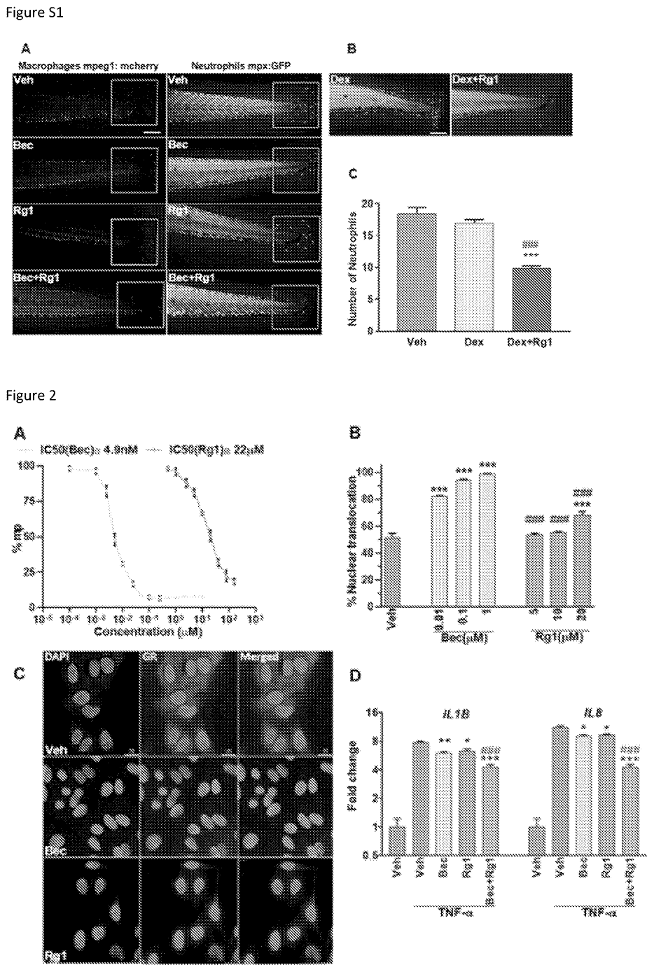

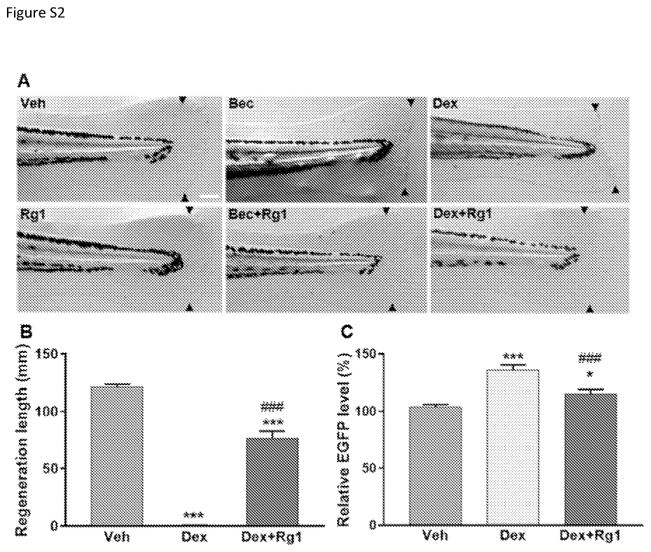

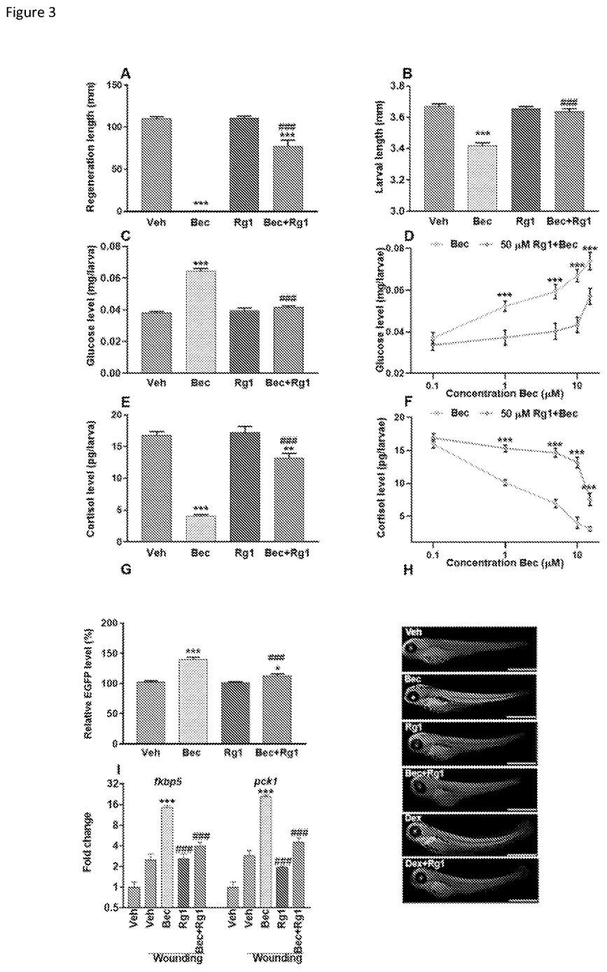

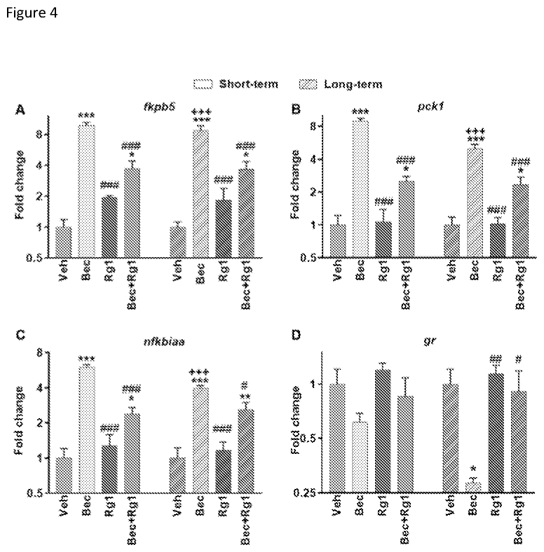

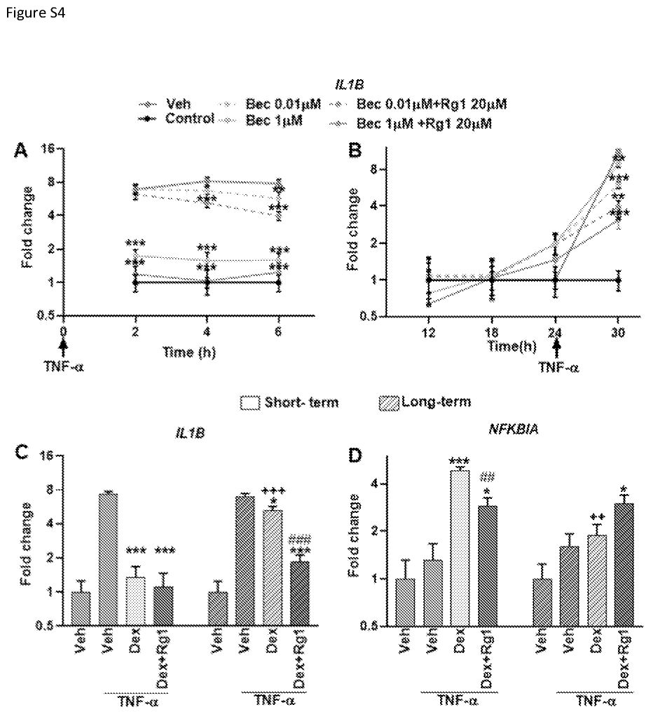

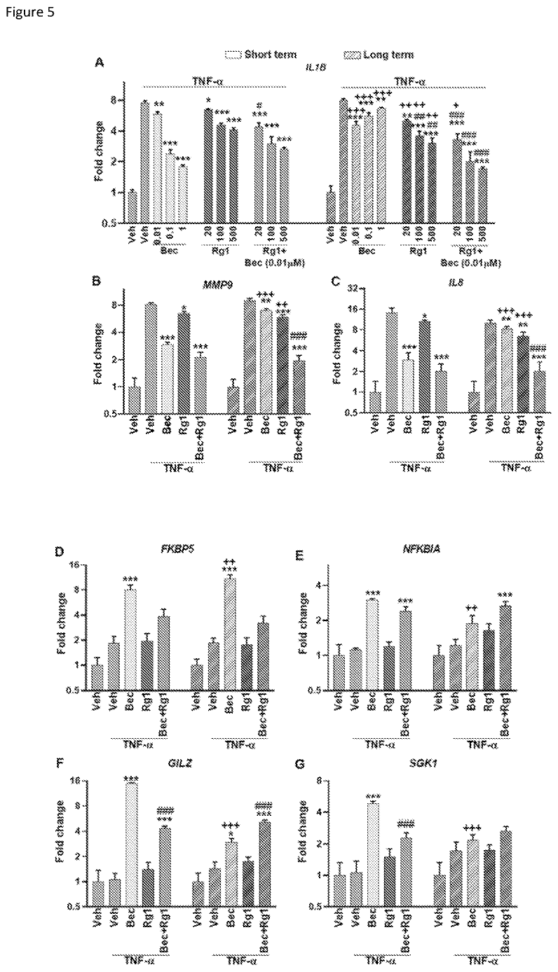

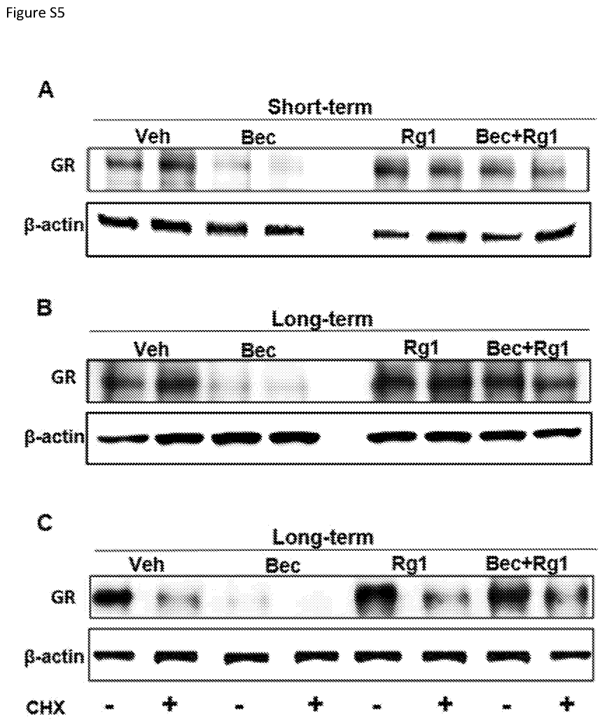

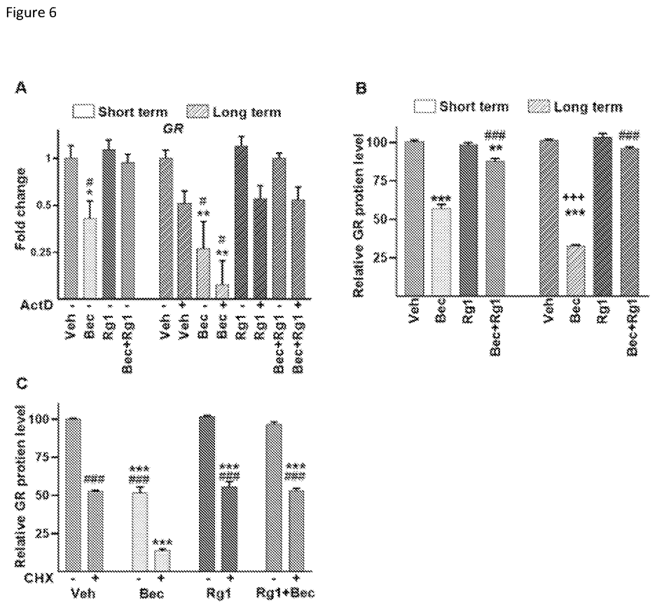

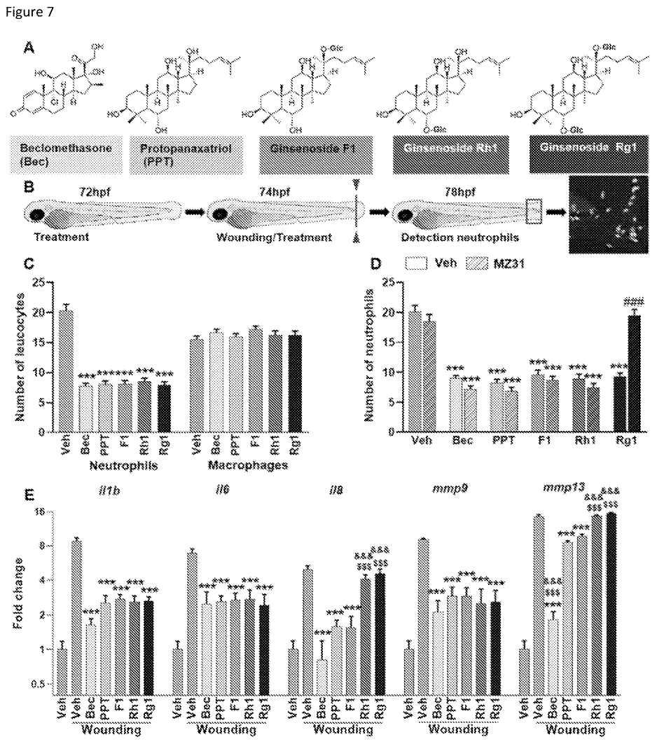

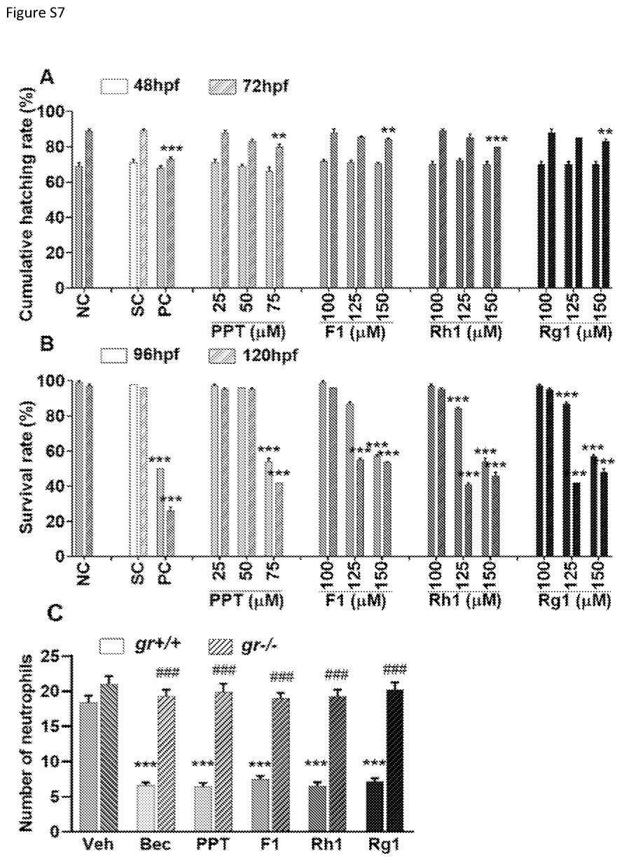

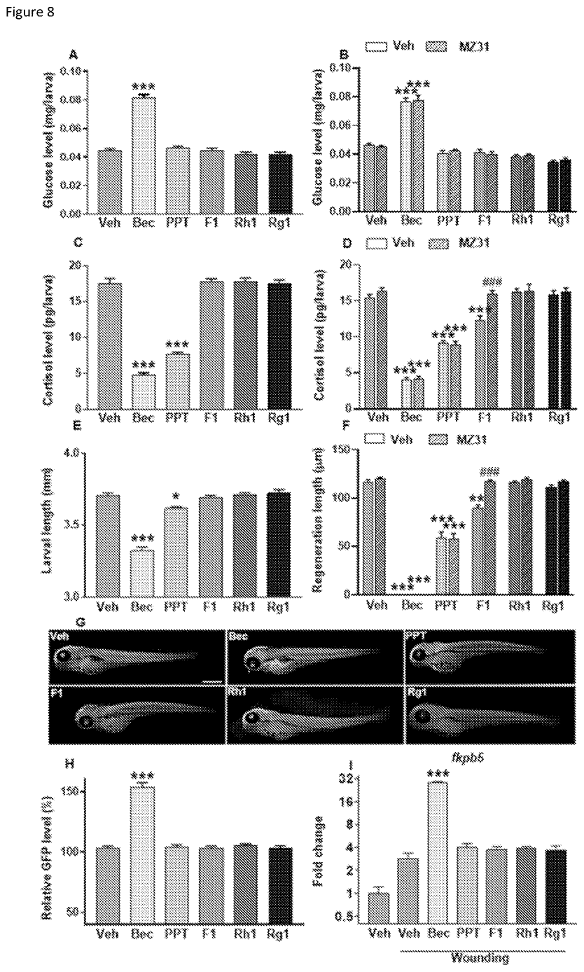

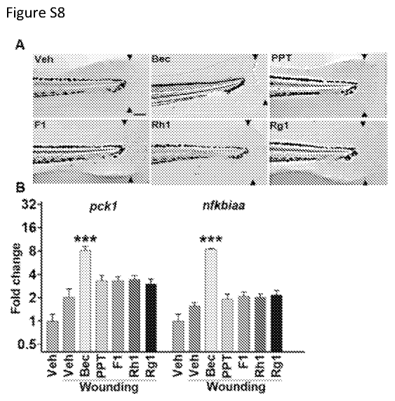

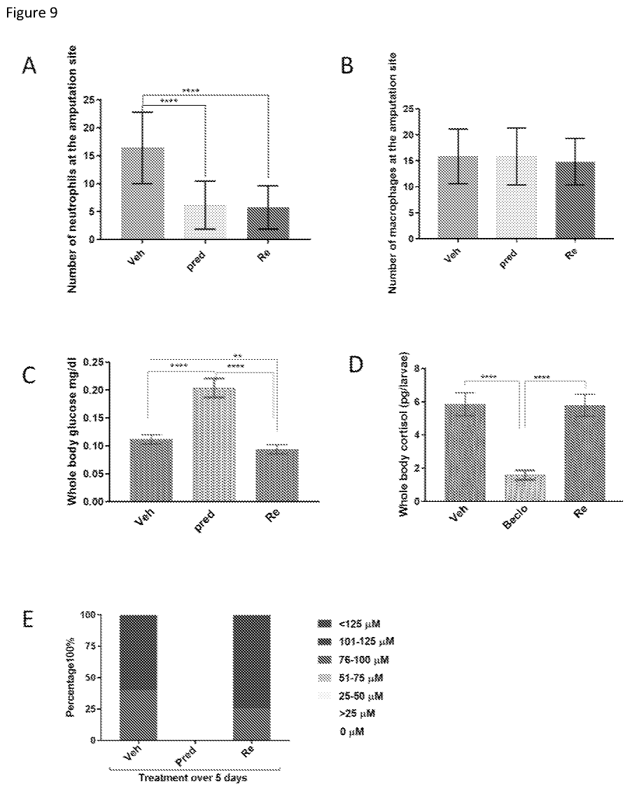

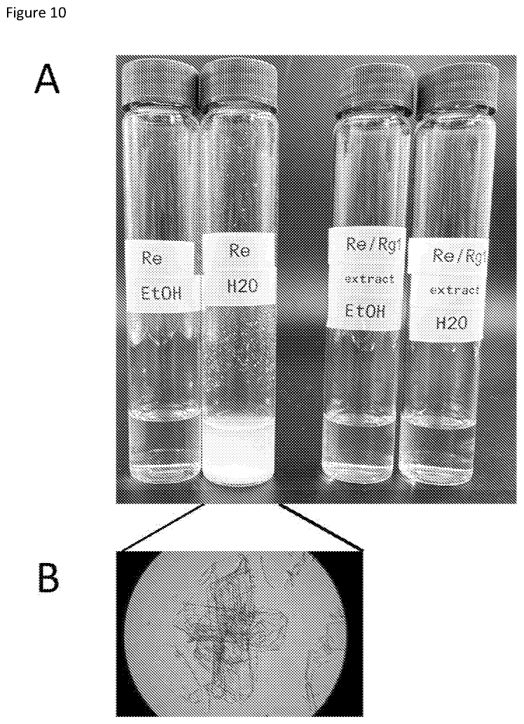

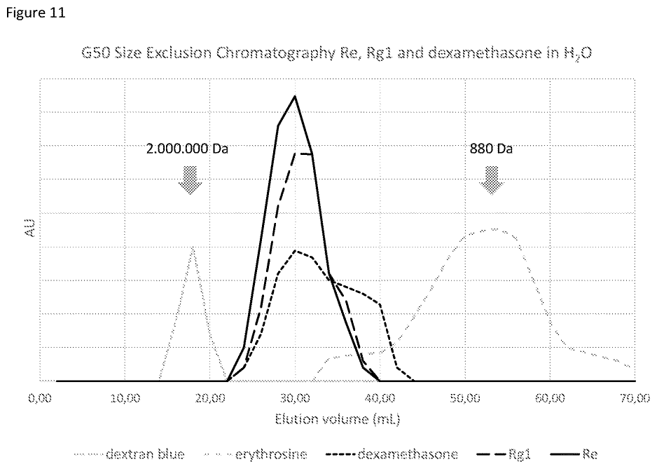

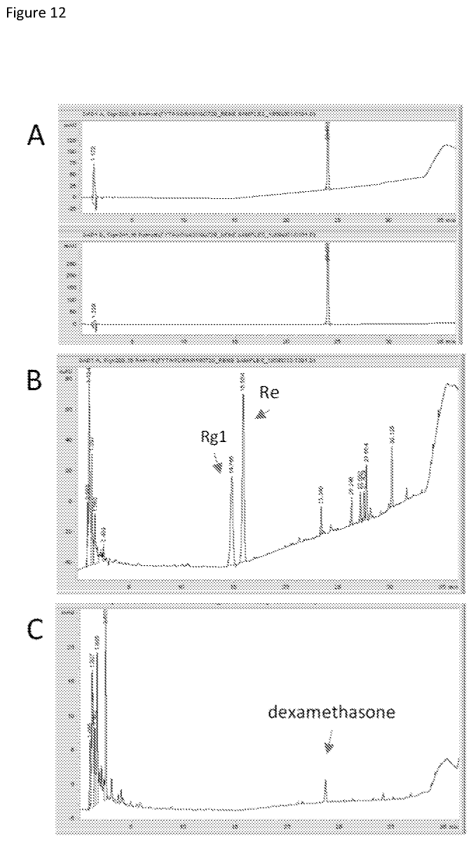

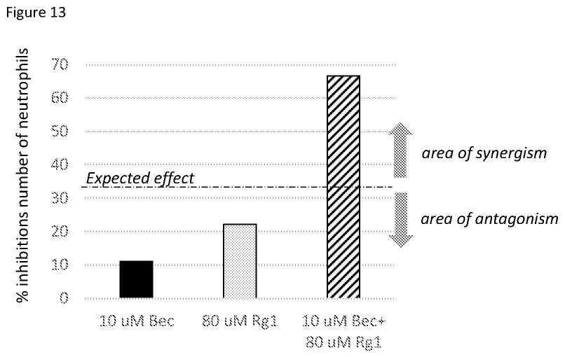

Rg1 is a PPT-type ginsenoside with molecular formula C₄₂H₇₂O₁₄ and a molecular weight of 801 g/mol. It is also known under the chemical names (3β,6α, 12β)-3,12-Dihydroxydammar-24-ene-6,20-diyl bis-β-D-glucopyranoside, Ginsenoside A2, Ginsenoside g1, Panaxoside A, Panaxoside Rg1, Sanchinoside C1 and Sanchinoside Rg1. The molecular structure of Rg1 is depicted in FIG. 1A.

Further, Re is another PPT-type ginsenoside and has molecular formula C₄₈H₈₂O₁₈ and a molecular weight of 947.15 g/mol. It is also known under the chemical names (3β, 6α, 12β)-20-(β-D-Glucopyranosyloxy)-3, 12-dihydroxydammar-24-en-6-yl 2-O-(6-deoxy-α-L-mannopyranosyl)-β-D-glucopyranoside, Chikusetsusaponin IVc, Ginsenoside B2, NSC 308877, Panaxoside Re and Sanchinoside Re.

Preferably, said ginsenoside is selected from Re, Rf, Rg1, Rg2, Rh1 or combinations thereof, more preferably said ginsenoside comprises Rg1, Re, or a combination of Rg1 and Re, more preferably, if said ginsenoside comprises a combination of Re and Rg1, said Rg1 and Re are present in a wt. ratio of between about 1:2 and about 2:1, more preferably between about 1:1.5 and about 1.5:1.

Said ginsenoside may be obtained from a natural source, or may be synthetically prepared.

For example, said ginsenoside may be obtained from the plant genus Panax. Preferably, said ginsenoside may be obtained from one or more of the species Panax bipinnatifidus, Panax, elegantior, P. ginseng, P. japonicus, P. major, P. notoginseng, P. omeiensis, P. pseudoginseng, P. quinquefolius, P. sikkimensis, P. sinensis, P. stipuleanatus, P. trifolius, P. vietnamensis, P. wangianus and P. zingiberensis.

Most preferably, said ginsenoside may be obtained from P. quinquefolius (also referred to as American ginseng), P. japonicus (also referred to as Japanese ginseng), P. ginseng (also referred to as Korean ginseng) or P. notoginseng (also referred to as South China ginseng).

Preferably, said ginsenoside may be obtained from one or more of the roots, the stems, the fruits, the rhizomes, the flowers or the leaves of a plant of the genus Panax, more preferably from one or more of the roots, stems, fruits, rhizomes, flowers or leaves of P. ginseng, P. japonicus, P. notoginseng or P. quinquefolius.

In a plant of the genus Panax, the content of ginsenosides is typically in the range of about 1 to about 10 wt. %, based on the dry weight of the plant, such as between about 2 and about 8 wt. %, between about 3 and about 6 wt. %, in particular about 4 wt. %, based on the dry weight of the plant.

Said ginsenoside may be isolated from a plant of the genus Panax, using any suitable method known in the art. For example, said ginsenoside may be obtained by drying a part, such as a root of a plant of the genus Panax, followed by extraction of the dried plant or plant part with a suitable solvent. Methods for preparing a ginseng extract is described in Chinese pharmacopeia edition 2015 (English language), volume I, page 524-526.

Optionally, said plant part, such as a root of a plant of Panax is steamed at 100° C. for at least two hours prior to drying. An extract obtained using this method is typically referred to as “red ginseng”.

Ginseng extracts are also commercially available, for example G115 from P. ginseng (Pharmaton SA, Switzerland) and NAGE from P. quinquefolius (Canadian Phytopharmaceuticals Corporation, Canada).

Preferably, the content of ginsenosides in a ginseng extract is at least 1 wt. %, more preferably at least 2 wt. %, at least 3 wt. %, at least 4 wt. %. Typically, the content of ginsenosides in a ginseng extract is at most 99 wt. %, more preferably at most 98 wt. %, even more preferably at most 95 wt. %

A preferred range of ginsenosides in a ginseng extract is between about 4 and about 50 wt. %, more preferably between about 8 wt. % and about 20 wt. %, based on the dry weight of the ginseng extract.

In an embodiment, said ginsenoside is provided as a ginseng extract.

Said ginseng extract optionally contains one or more components other than ginsenosides, such as ginseng proteins and ginseng carbohydrates.

Alternatively, said ginsenoside is provided in isolated form, i.e. essentially separated from components naturally present in a plant from the genus Panax. Isolated ginsenosides are commercially available, for example Ginsenoside-Rb1 (CAS 41753-43-9), Ginsenoside-Rh1 (CAS 63223-86-9), Ginsenoside Re (CAS 52286-59-6) and Ginsenoside Rg1 (CAS 22427-39-0) are commercially available from Sigma Aldrich.

An isolated ginsenoside may also be optionally provided in synthetic form if a chemical synthetic pathway is known. For example, the chemical synthesis of ginsenoside Rg3 is described by Anufriev et al, Carbohydr Res. 1997; 304(2):179-182.

Preferably, the purity of said ginsenoside is at least 90 wt. %. More preferably, the purity of said ginsenoside is at least 92 wt. %, at least 94 wt. %, at least 96 wt. %, at least 98 wt. %, at least 99 wt. %, such as 100 wt. %.

### Micelle

The inventors further recognized that the solubility of ginsenosides in water was a limiting factor in the medical use of ginsenosides in the treatment of an inflammatory disease, or prevention of side effects caused by GC treatment or in the preparation of a pharmaceutical composition according to the invention. Further, ginsenosides notoriously suffer from degradation in the gastro-intestinal tract rendering them ineffective and hampering their systemic use.

The inventors therefore developed a novel formulation of ginsenosides in which one or more of the abovementioned drawbacks have been overcome.

Accordingly, said ginsenoside for use according to the invention is preferably at least partly incorporated into a micelle, more preferably in an outer layer thereof.

The inventors realized that when ginsenosides were formulated as a micelle, solubility in water could be markedly increased and stability in the intestinal tract was improved compared to a regular ginsenoside formulation, wherein the ginsenoside was not incorporated in (an outer layer of) a micelle. Such increased water solubility is beneficial as it allows to obtain a sufficiently high concentration of a ginsenoside in an aqueous medium, typically required to counteract one or more side-effects of GC treatment or obtain a an anti-inflammatory effect.

Further, improved stability in the intestinal tract advantageously improves efficacy of systemic use of said ginsenoside.

A micelle having at least one ginsenoside incorporated therein, preferably at least partly incorporated in an outer layer of the micelle, may in principle be prepared using any suitable method known in the art, depending on the type of ginsenoside to be incorporated into the micelle. For example, for a micelle having one or more PPD-type ginsenosides incorporated therein, said micelle may be prepared by slowly evaporating a solution of a PPD-type ginsenoside in an organic or aqueous solvent (e.g. ethanol or a mixture of ethanol and water) under reduced pressure, followed by slowly resolving the obtained film of ginsenosides in water to obtain a micelle having one or more PPD-type ginsenosides incorporated therein.

However, the inventors surprisingly realized that when this method was applied using the PPT-type ginsenoside Re, crystallization of Re was observed, but no micelle formation occurred. This is demonstrated in Example 4.

The inventors surprisingly realized that a micelle having at least one PPT-type ginsenoside incorporated therein could however be prepared using a (crude) ginsenoside extract comprising at least one PPT-type ginsenoside instead of an isolated PPT-type ginsenoside as substrate.

Accordingly, the invention further relates to a ginsenoside formulation comprising a PPT-type ginsenoside at least partly incorporated therein, preferably at least partly incorporated in an outer layer of the micelle, said micelle having a molecular weight of at least 10.000 Da.

Said ginsenoside is as defined herein above in the section “ginsenoside” provided it is a PPT-type ginsenoside.

The micelles have a molecular weight of at least 10.000 Da, preferably at least 20.000 Da, at least 40.000 Da, at least 50.000 Da, at least 60.000 Da, at least 70.000 Da, at least 80.000 Da, at last 90.000 Da, most preferably at least 100.000 Da.

The micelles typically have a molecular weight of at most 100.000.000 Da, preferably at most 10.000.000 Da, most preferably at most 1.000.000 Da.

The micelles preferably have a molecular weight in the range of between about 10.000 and about 100.000.000 Da, more preferably in the range of between about 50.000 and about 10.000.000 Da, even more preferably in the range of between about 80.000 and about 1.000.000 Da.

Said molecular weight may be determined using any suitable method known in the art, for example using size-exclusion chromatography or High-Performance Liquid Chromatography, in particular using the method as described in Example 4.

Typically, the micelles comprise between about 10 and about 100.000 molecules of one or more ginsenosides, more preferably between about 20 and about 10.000 molecules of one or more ginsenosides, even more preferably between about 50 and about 10.000 molecules of one or more ginsenosides, in particular between about 100 and about 1000 molecules of one or more ginsenosides. The solubility in an aqueous medium, preferably water, of a ginsenoside formulation according to the invention, or a ginsenoside which is at least partly incorporated in (an outer layer of) a micelle, is preferably at least 0.03 mmol/L, more preferably at least 0.05 mmol/L, at least 0.1 mmol/L, at least 1 mmol/L, at least 5 mmol/L, at least 10 mmol/L, at least 50 mmol/L, at least 0.1 mol/L, at least 0.15 mol/L, at least 0.2 mol/L, at least 0.25 mol/L, most preferably at least 0.3 mol/L.

Typically, the solubility in an aqueous medium, preferably water, of a ginsenoside formulation according to the invention, or a ginsenoside which is at least partly incorporated in (an outer layer of) a micelle is in the range of between about 0.03 mmol/L and about 10 mol/L, preferably between about 0.05 mmol/L and about 5 mol/L, more preferably between about 0.1 mmol/L and about 2 mol/L, between about 1 mmol/L and about 1 mol/L, even more preferably between about 5 mmol/L and about 0.5 mol/L, in particular between about 10 mmol/L and about 0.3 mol/L.

The solubility in an aqueous medium, preferably water, of a ginsenoside formulation according to the invention, or a ginsenoside which is at least partly incorporated in (an outer layer of) a micelle is preferably at least 30 mg/L, more preferably at least 50 mg/L, at least 100 mg/L, at least 1 g/L, at least 5 g/L, at least 10 g/L, at least 50 g/L, at least 100 g/L, at least 150 g/L, at least 200 g/L, at least 250 g/L, most preferably at least 300 g/L.

The solubility in an aqueous medium, preferably water, of a ginsenoside formulation according to the invention, or a ginsenoside which is at least partly incorporated in (an outer layer of) a micelle, is preferably in the range of between about 30 mg/L and about 1000 g/L, more preferably between about 50 mg/L and about 5000 g/L, more preferably between about 100 mg/L and about 2000 g/L, even more preferably between about 1 g/L and about 1000 g/L, between about 5 g/L and about 500 g/L, in particular between about 10 g/L and about 300 g/L.

Particularly good results have been obtained with a ginsenoside formulation according to the invention or a ginsenoside which is at least partly incorporated in (an outer layer of) a micelle, comprising Rg1 and/or Re which are at least partly incorporated into said micelle, preferably wherein Rg1 and Re are present in a wt. ratio of between about 1:2 and about 2:1, more preferably between about 1:1 and about 1:1.5. Preferably, said micelle has a molecular weight of between about 50.000 and about 100.000 Da.

The invention further relates to a method for preparing a ginsenoside formulation according to the invention or a ginsenoside which is at least partly incorporated in (an outer layer of) a micelle, said method comprising:
(a) providing a ginsenoside extract from a plant or plant part of the genus Panax, preferably Panax ginseng;
(b) contacting said ginsenoside extract with an aqueous solvent mixture to obtain a ginsenoside mixture;
(c) at least partly removing said aqueous solvent mixture from said ginsenoside mixture; and optionally
(d) reconstituting the ginsenoside mixture obtained in step c) in water to obtain a ginsenoside formulation according to the invention.

Said ginsenoside extract may be obtained using any suitable method known in the art, for example by boiling a dried plant or plant part from the genus Panax in water, followed by filtering the water phase to obtain a crude ginsenoside extract.

The amount of water to be used herein is dependent on the amount of dried plant or dried plant parts to be extracted. It is within the ability of the skilled person to select appropriate amounts of water, based on his common general knowledge and the information provided herein.

Preferably, said ginsenoside extract is subjected to one or more purification steps prior to or during contacting the ginsenoside extract with said aqueous solvent mixture in step b. Preferably, said ginsenoside extract is subjected to a step of column chromatography using a non-polar resin as stationary phase.

Said non-polar resin is preferably a D101-type resin, a D201-tpe resin, a D113-type resin, a D285-type resin, a D296-type resin, a D941-type resin, a D945-type resin, a DM130-type resin, a DM131-type resin, a Dt-type resin, a HPD100-type resin, a HPD300-type resin, an NKA-type resin, an LK37-type resin, an LK1300S-type resin, an LK20-type resin, a 388-type resin, Amberlite IRA900, an AB-8-type resin, Amberlite XAD16, an SA-2-type resin or an LX-TS4-type resin.

Said ginsenoside is preferably eluted from said non-polar resin with a suitable aqueous solvent mixture to obtain a purified ginsenoside extract. Said aqueous solvent mixture preferably is a mixture of water and a non-aqueous solvent. Preferably said non-aqueous solvent has a boiling point lower than water. Preferably, said aqueous solvent mixture is a mixture of alcohol in water, such as between about 40% and about 80% alcohol in water. More preferably, said aqueous solvent is a mixture of methanol, ethanol, propanol or isopropanol in water, most preferably a mixture of between about 40% and about 80% of ethanol in water.

Said non-polar resin is preferably first washed with a polar solvent, preferably water, prior to elution of the ginsenoside from the non-polar resin using the aqueous solvent mixture as defined herein above.

Said ginsenoside extract is preferably subjected to a step of decolorization. Preferably said purified ginsenoside extract is contacted with a decolorization agent, preferably a D941 macroporous resin. Preferably said ginsenoside extract is subjected to a step of decolorization after having been subjected to contacting with a non-polar resin. Preferably, said step of decolorization is carried out in step b), e.g. said ginsenoside extract is contacted with a an aqueous solvent mixture and said aqueous solvent mixture comprising said ginsenoside extract is contacted with a decolorization agent.

The amount of solvents, non-polar resin and decolorization agent to be used herein is dependent on the amount of ginsenoside extract to be subjected to the method. It is within the ability of the skilled person to select appropriate amounts of non-aqueous solvent, polar solvent, non-polar resin and decolorization agent, based on his common general knowledge and the information provided herein.

Said aqueous solvent mixture is preferably removed from said ginsenoside mixture in step c. This can be achieved using any suitable method known in the art, preferably by evaporation under reduced pressure. Optionally, said ginsenoside mixture may be heated, typically to a temperature between 30° C. and 90° C., such as between 40° C. and about 80° C. to aid evaporation of (part of) the aqueous solvent mixture.

Said ginsenoside mixture obtained in step c) is preferably dissolved in water to obtain a ginsenoside formulation according to the invention.

Alternatively, said non-aqueous solvent present in said aqueous solvent mixture is at least partly evaporated to obtain a water fraction or an aqueous solvent mixture enriched in water, comprising a ginsenoside formulation according to the invention.

Said ginsenoside in said ginsenoside formulation according to the invention is at least partly incorporated into a micelle, preferably at least partly incorporated in an outer layer of a micelle. This can be determined using any suitable analytic method known in the art, for example size exclusion chromatography, optionally in combination with HPLC, e.g. as described in Example 4.

Alternatively or additionally, said micelle preferably further comprises a GC, more preferably selected from dexamethasone and beclomethasone, at least partly incorporated therein. In this embodiment, said ginsenoside is preferably incorporated in an outer layer of a micelle and said GC is preferably present in an inner cavity of the micelle. Said micelle may be prepared using the method as described herein above, provided said GC is preferably contacted with said ginsenoside extract in step b).

The invention further relates to a ginsenoside formulation obtainable by method according to the invention.

Said ginsenoside formulation preferably comprises a PPT-type ginsenoside at least partly incorporated in a micelle, preferably at least partly incorporated in an outer layer of the micelle, said micelle having a molecular weight of at least 10.000 Da and/or a molecular weight of at most 100.000 Da.

Further, said ginsenoside is preferably a PPT-type ginsenoside, preferably selected from Rg1, Re and a combination thereof.

### Glucocorticoid

As the skilled person is aware, various types of GC's are known in the art. Examples include short-acting GC's, such as cortisol or cortisone, intermediate-acting GC's, such as prednisone, long-acting GC's such as dexamethasone and betamethasone and mineralocorticoids such as fludrocortisone.

Said GC in a combination for use according to the invention may be any natural or synthetic GC, or a pro-drug thereof. Preferably, said GC is selected from dexamethasone, betamethasone, prednisolone, methylprednisolone, triamcinolone, deflazacort, fludrocortisone acetate, desoxycorticosterone acetate, aldosterone, hydrocortisone and beclomethasone or a prodrug thereof, preferably a prodrug selected from cortisone or prednisone.

In an embodiment, said GC is preferably a long-acting GC. Alternatively or additionally, said GC is beclomethasone or dexamethasone.

Advantageously, a combination for use according to the invention allows to apply GCs in a treatment which are normally associated with very severe side-effects and therefore typically not administered to a subject. Such GCs may however be applied in a combination for use according to the invention, due to the presence of the ginsenoside to mediate the side-effects. This advantageously expands the options for treatment of an inflammatory disease in a subject in need thereof.

### Medical Use

Said side-effect of GC treatment may be any side effect that is induced by (chronic) use of one or more GC('s). Examples of side-effects that may occur include diabetes, decreased cortisol levels, reduced growth velocity, wound healing disorders, tissue degeneration, osteoporosis, hypertension, weight gain and muscle weakness.

Preferably, said side-effect of GC treatment is selected from decreased cortisol levels, reduced growth velocity and wound healing disorders.

Accordingly, in another aspect the invention relates to a ginsenoside for use in a method of prevention or treatment of (GC-induced) diabetes, (GC-induced) decreased cortisol levels, (GC-induced) reduced growth velocity, (GC-induced) wound healing disorders, GC-induced tissue degeneration, (GC-induced) osteoporosis, (GC-induced) hypertension, (GC-induced) weight gain and (GC-induced) muscle weakness, said method comprising administering a ginsenoside to a subject that is suffering from, or at risk of suffering from, one or more side-effect(s) of GC treatment. Preferably, the invention relates to a ginsenoside for use in a method of prevention or treatment of (GC-induced) decreased cortisol levels, (GC-induced) reduced growth velocity and (GC-induced) wound healing disorders.

Said subject may be any subject that suffers from GC-induced side-effects, preferably a mammal, more preferably a human.

In particular, said subject is a subject that is subjected to or has been subjected to GC treatment. Preferably, said subject is subjected to a daily dosage of GC.

Typically, said subject is subjected to long-term treatment with one or more GC(s), such as during a period of at least 2 days, preferably at least 7 days, preferably at least 14 days, more preferably at least 21 days, more preferably at least 28 days, more preferably at least 35 days, more preferably at least 6 weeks, preferably at least 8 weeks, more preferably at least 12 weeks, more preferably at least 16 weeks, at least 20 weeks, at least 25 weeks, at least 30 weeks, at least 35 weeks, at least 40 weeks, at least 45 weeks, at least 50 weeks, more preferably at least one year, at least 1.5 year, at least 2 years, at least 2.5 years, at least 3 years, at least 3.5 years, at least 5 years, most preferably for at least 10 years.

Typically, said subject has been subjected to treatment with one or more GC(s) during a period of between about 6 weeks and about 10 years, between about 8 weeks and about 5 years, between about 12 weeks and about 3 years, between about 16 weeks and about 1 year.

Preferably, said subject is under treatment with one or more GC(s) simultaneously with said treatment of a side-effect of GC treatment. Alternatively, said subject has been subjected to GC treatment prior to said treatment of said side-effect. Preferably said treatment with one or more GC(s) is less than 5 years ago, more preferably less than 4 years, less than 3 years, less than 2 years, less than 1 year, less than 9 months, less than 6 months, less than 4 months, less than 2 months, most preferably less than 1 month prior to said treatment of said side-effect of GC treatment.

Typically, said treatment with one or more GC(s) is between about 1 week and about 5 years prior to said treatment of said side-effects of GC treatment, preferably between about 2 weeks and about 4 years, between about 4 weeks and about 3 years, between about 2 months and about 2 years, such as between about 4 months and about 1 year.

A typical dosage for GC treatment varies between about 0.5 mg to about 150 mg, such as between about 1 mg and about 100 mg of GC, in particular between about 4 mg and about 50 mg, preferably about 5 mg to about 20 mg. Usually, the dose of GC is between about 0.007 to about 2 mg/kg, such as between 0.01 and about 1.5 mg/kg, in particular between about 0.05 mg/kg and about 0.7 mg/kg, preferably between about 0.07 mg/kg and about 0.3 mg/kg. Typically, the dose of GC is between about 0.2 mg/m2 body surface area and about 85 mg/m2 body surface area, preferably between about 0.5 mg/m2 and about 60 mg/m2, more preferably between about 2 mg/m2 and about 30 mg/m2, such as between about 3 mg/m2 and about 15 mg/m2.

As the skilled person will appreciate, said dosage of said ginsenoside depends on the circumstances of the case, such as the patient, the side-effect to be treated and the dosage of GC the subject is being subjected to or was subjected to. A typical dose of ginsenoside is between about 4 mg and about 400 mg, such as between about 30 mg and about 70 mg. Usually, the dose of GC is about 0.05 to about 5.5 mg/kg, such as between about 0.4 and about 1.0 mg/kg.

Said dosage of ginsenoside may be provided to a subject in a single dose, or may be provided in multiple doses per day, such as between 2 to 4 doses per day, provided the total amount administered per day is the same.

Preferably, in particular in case a subject is subjected to GC treatment, said ginsenoside is administered to said subject in a molar amount that is at least equal to the amount of GC. More preferably, said ginsenoside is administered in a molar excess, with regards to GC, preferably a molar excess of at least 4, more preferably at least 8, even more preferably at least 10, at least 20, at least 50, at least 100, at least 1000, at least 2000.

Preferably, said ginsenoside is administered to said subject in a molar ratio, with regards to GC of between about 1 (GC) to 2000 (ginsenoside), preferably between about 4 to about 1000, about 6 to about 100, about 8 to about 20.

Said ginsenoside may be administered in any suitable way. Said ginsenoside may be administered topically, enterally or parenterally. Further, said ginsenoside may be administered systemically, such as by oral administration, sublingual administration, buccal administration or rectal administration. Alternatively or additionally, said combination may be administered locally, such as transdermal, or via (oral) inhalation or via transnasal administration. Said combination may also be administered by means of injection, e.g. subcutaneously, intravenously or intramuscularly. Preferably, said ginsenoside is administered orally or transdermally.

Said ginsenoside is preferably administered in a formulation with a suitable carrier, preferably an aqueous carrier, more preferably a hydrogel. In a specific embodiment, said ginsenoside is at least partly incorporated into a micelle, preferably in an outer layer of said micelle, said micelle having a molecular weight of at least 10.000 Da and is administered in a formulation comprising an aqueous carrier. Such a ginsenoside was particularly effective in treatment or prevention of GC treatment.

The invention further relates to a method of treatment of a side-effect of GC treatment, preferably (GC-induced) diabetes, (GC-induced) decreased cortisol levels, (GC-induced) reduced growth velocity, (GC-induced) wound healing disorders, (GC-induced) tissue degeneration, (GC-induced) osteoporosis, (GC-induced) hypertension, (GC-induced) weight gain and (GC-induced) muscle weakness, comprising administering a ginsenoside to a subject that is suffering from, or at risk of suffering from, one or more side-effect(s) of GC treatment.

The invention further relates to a use of ginsenoside for the preparation of a medicament for the treatment of a side-effect of GC treatment, preferably (GC-induced) diabetes, (GC-induced) decreased cortisol levels, (GC-induced) reduced growth velocity, (GC-induced) wound healing disorders, (GC-induced) tissue degeneration, (GC-induced) osteoporosis, (GC-induced) hypertension, (GC-induced) weight gain and (GC-induced) muscle weakness.

### Combination of Ginsenoside and GC for Use in a Method of Prevention or Treatment of an Inflammatory Disease

The inventors further realized that co-treatment of a ginsenoside and a GC has several advantages over the individual use of a GC or a ginsenoside as anti-inflammatory medicaments.

For example, a ginsenoside advantageously reduces resistance against said GC treatment. Without wishing to be bound by any theory, it is believed that a ginsenoside advantageously stabilizes the receptor mRNA and the receptor protein and accordingly prevents down regulation of the GR, thereby reducing sensitivity to GC treatment (Example 1).

In addition, the inventors found that a ginsenoside and a GC advantageously exhibit a synergistic anti-inflammatory effect. This is advantageous, as lower dosages of GC and ginsenosides are required, whilst still achieving an effective anti-inflammatory response (FIG. 1c). Using a lower dosage of GC may also advantageously decrease the risk of obtaining one or more side-effects typically associated with GC treatment.

Accordingly, the invention further pertains to a combination of a GC and a ginsenoside for use in a method of prevention or treatment of an anti-inflammatory disease. The inventors further realized that the side-effects of GC treatment may be effectively prevented or treated when a GC and a ginsenoside are administered in a specific molar ratio, whilst maintaining an anti-inflammatory effect.

Without wishing to be bound by any theory, the inventors believe that said ginsenoside acts as a competitive antagonist on the glucocorticoid receptor by inhibiting dimerization of the glucocorticoid receptor (see Example 1). Accordingly, the invention relates to a combination of a GC and a ginsenoside for use in a method of prevention or treatment of an anti-inflammatory disease, wherein said method comprises administering a GC and a ginsenoside in a molar ratio of between about 1:1 and about 1:2000, preferably between about 1:4 and about 1:1000, more preferably between 1:8 and 1:20 to a subject in need thereof.

Said ginsenoside and said GC is as described herein above.

Preferably, said molar ratio between said GC and said ginsenoside is between about 1:2 and about 1:1000, more preferably between 1:4 and 1:500, more preferably between about 1:6 and about 1:250, even more preferably between about 1:7 and about 1:100, in particular between about 1:8 and about 1:20. Without wishing to be bound by any theory it is believed that within these molar ranges, optimal competitive antagonistic effect of said ginsenoside is obtained. Further, at these molar ranges, solubility of said ginsenoside at a therapeutically effective amount is feasible, especially when said ginsenoside is administered in a micelle formulation, as described herein above.

Said GC and said ginsenoside may be administered together, or separately. Accordingly, in an embodiment, said GC and ginsenoside are administered as a pharmaceutical composition according to the invention.

Alternatively, said GC and said ginsenoside may be administered separately. Said GC and ginsenoside are preferably administered within a time window of 1 hour or less, preferably within 45 minutes or less, such as within 30 minutes or less, in particular within 15 minutes or less. Most preferably, said GC and ginsenoside are administered essentially at the same time.

Alternatively, said ginsenoside may be administered slightly before or after the GC, for example approximately 60 minutes before or after administering the GC, preferably approximately 45 minutes before or after administering the GC, more preferably approximately 30 minutes before or after administering the GC, such as 15 minutes before or after administering the GC.

As mentioned herein, said GC and ginsenoside advantageously act synergistically together to achieve an anti-inflammatory effect. This synergistic effect allows administration of GC and ginsenoside at a lower dosage than typically required to induce an anti-inflammatory effect, if the GC and ginsenoside are administered in separate treatments.

Accordingly, the invention preferably relates to a combination of a GC and a ginsenoside for use according to the invention, wherein said GC and/or said ginsenoside are administered in a therapeutically ineffective amount. In other words, said GC and/or said ginsenoside are preferably administered in an amount that is lower than the amount of GC or ginsenoside that would be administered in a mono-treatment of GC or a ginsenoside to the same subject in need thereof for the treatment of the same anti-inflammatory disease.

Preferably, said GC and/or said ginsenoside is administered in an amount that is at most 80% of the therapeutically effective amount, more preferably at most 75%, at most 70%, at most 65%, at most 60%, at most 55%, at most 50%, at most 45%, at most 40%, at most 35%, at most 30%, at most 25%, at most 20%, at most 15%, at most 10%, at most 5%. Preferably, said GC and/or said ginsenoside is administered in an amount that is between about 1% and about 80% of the therapeutically effective amount, more preferably between about 2% and about 75%, between about 3% and about 70%, between about 4% and about 65%, between about 5% and about 60%, such as between about 10% and about 50% of the therapeutically effective amount.

As the skilled person will appreciate, the dose of GC or ginsenoside that would normally be prescribed to a subject depends on the patient, the age of the patient, the illness, the type of GC or ginsenoside, the dosage form and the circumstances. For example, the severity of the illness may require a higher dosing to be effective. Likewise, as described herein, a patient which has been subjected to long term use of GC's may become at least partially resistant to GC treatment, which also usually requires higher dosing to achieve an effect. Further, a systemically applied GC is typically administered in a different dosage than a locally applied GC.

The skilled person is capable of selecting a suitable dosage (which preferably corresponds to a therapeutically ineffective amount), based on the information provided herein, common general knowledge and a reasonable amount of trial and error.

Typical dosages of GC's frequently administered can be found in medical registers, such as for example the Dutch “geneesmiddeleninformatiebank” (https://www.geneesmiddeleninformatiebank.nl/ords/f?p=111:1:0::NO:RP,1:P0_DO MAIN, P0_LANG:H, EN; accessed on May 7 2024).

As a rule of thumb, a 5 mg prednisone dose is equivalent to a dose of about 0.75 mg dexamethasone, 0.6 mg betamethasone, 4 mg methylprednisolone, 4 mg triamcinolone, 5 mg prednisolone, 20 mg hydrocortisone or 25 mg cortisone, 2 mg fludrocortisone (Nicolaides N C, Pavlaki A N, Maria Alexandra M A, et al. Glucocorticoid Therapy and Adrenal Suppression. [Updated 2018 Oct. 19]. In: Feingold K R, Anawalt B, Blackman M R, et al., editors. Endotext [Internet]. South Dartmouth (MA): MDText.com, Inc.; 2000-. Table 1: [Glucocorticoid Equivalencies ( )]. Available from: https://www.ncbi.nlm.nih.gov/books/NBK279156/table/adrenal_glucocorticoid-therapy-and-adrenal-suppression; accessed on May 7 2024)

Common dosages for GC's typically administered to a subject in need thereof are furthermore shown in the table below. Herein, the dosage range in mg/kg relates to the total kg of body weight of the subject tin need thereof. Herein, the dosage range in mg/m2 body surface relates to the total m2 body surface of the subject in need thereof. As the skilled person will appreciate, a GC that is administered locally, such as topically on the skin, the local concentration of GC applied to the infected area of the skin may exceed the concentration as shown in the table below.

| | Dosage range | Dosage range | Dosage range |
|---|---|---|---|
| GC | (mg) | (mg/kg) | (mg/m²) |
| Dexamethasone | 0.5-10 | 0.01-0.13 | 0.3-5 |
| Betamethasone | 0.5-7 | 0.008-0.1 | 0.3-4 |
| Prednisone | 5-200 | 0.07-2.8 | 2.5-105 |
| Prednisolone | 5-30 | 0.07-0.40 | 2.5-15 |
| Methylprednisolone | 4-50 | 0.06-0.7 | 2-30 |
| Triamcinolone | 0.2-35 | 0.003-0.46 | 0.1-29 |
| Hydrocortisone | 20-800 | 0.28-11.2 | 10-420 |
| Cortisone | 25-300 | 0.36-4.3 | 10-200 |
| Fludrocortisone | 2-25 | 0.03-0.3 | 1-15 |
| Beclomethasone | 0.05-0.35 | 0.001-0.005 | 0.03-0.2 |

Accordingly, a typical dosage for GC in a combination of GC and ginsenoside (for use in a method) according to the invention varies between about 0.01 mg to about 150 mg, preferably between about 0.1 mg to about 100 mg, more preferably between about 0.5 mg to about 50 mg, most preferably between about 1 mg and about 20 mg of GC.

Preferably, the dose of GC is about 0.001 mg/kg to about 2.5 mg/kg, preferably between about 0.007 to about 1.5 mg/kg, more preferably between about 0.014 mg/kg and about 0.7 mg/kg, most preferably between 0.03 and about 0.3 mg/kg. Preferably, the dose is at most 2.5 mg/kg, more preferably at most 1.4 mg/kg, at most 0.7 mg/kg, at most 0.3 mg/kg, at most 0.07 mg/kg, most preferably at most 0.007 mg/kg, in particular at most 0.0001 mg/kg. Preferably, the dose of GC is about 0.01 mg/m2 of body surface area to about 100 mg/m2 of body surface area, preferably between about 0.05 mg/m2 of body surface area and about 60 mg/m2 of body surface area, more preferably between about 0.1 mg/m2 of body surface area and about 30 mg/m2 of body surface area, most preferably between about 0.2 mg/m2 of body surface area and about 15 mg/m2 of body surface area. Preferably, the dose of GC is at most about 60 mg/m2 of body surface area, more preferably at most about 30 mg/m2 of body surface area, more preferably at most about 15 mg/m2 of body surface area, even more preferably at most about 5 mg/m2 of body surface area, such as at most 2 mg/m2 of body surface area, more preferably at most about 1 mg/m2 of body surface area, most preferably at most about 0.2 mg/m2 of body surface area.

Said dosage may be provided to a subject in a single dose, or may be provided in multiple doses per day, such as between 2 to 4 doses per day, provided the total amount administered per day is approximately the same.

Accordingly, the invention preferably relates to a combination of GC and ginsenoside for use according to the invention, wherein said GC is administered at a dose of at most about 150 mg/day, preferably at most 100 mg/day, more preferably at most 50 mg/day, more preferably at most 25 mg/day, more preferably at most 10 mg/day, more preferably at most 5 mg/day, even more preferably at most 4 mg/day, at most 3 mg/day, at most 2 mg/day, at most 1 mg/day, even more preferably at most 0.5 mg/day, in particular at most 0.1 mg/day, more in particular at most 0.01 mg/day. Preferably, said GC is administered at a dose of between about 0.001 mg/day and about 150 mg/day, preferably between about 0.01 mg/day and about 100 mg/day, more preferably between about 0.1 mg and about 50 mg/day, most preferably between about 0.5 mg and about 20 mg/day.

In a preferred embodiment, the invention relates to a combination of a GC and a ginsenoside for use according to the invention, wherein said GC and said dosage thereof is as in the table provided below.

| | | | Dosage (mg/m² |
|---|---|---|---|
| | | Dosage (mg/kg | of body surface |
| GC | Dosage (mg) | body weight) | area) |
| Dexamethasone | 0.5-5 | 0.007-0.07 | 0.25-2.5 |
| Betamethasone | 0.3-3.5 | 0.005-0.05 | 0.2-2 |
| Prednisone | 3.5-35 | 0.05-0.5 | 2-22 |
| Prednisolone | 3.5-35 | 0.05-0.5 | 2-22 |
| Methylprednisolone | 2.5-30 | 0.04-0.4 | 1.4-15 |
| Triamcinolone | 2.5-30 | 0.04-0.4 | 1.4-15 |
| Hydrocortisone | 10-100 | 0.15-1.5 | 5-60 |
| Cortisone | 15-150 | 0.22-2 | 8-80 |
| Fludrocortisone | 1.5-15 | 0.02-0.2 | 0.7-8 |
| Beclomethasone | 0.001-0.3 | 0.001-0.005 | 0.03-0.2 |

Accordingly, in a particularly preferred embodiment, the invention relates to a combination of a GC and a ginsenoside for use in a method according to the invention, wherein said GC is dexamethasone, and wherein said GC is administered in a therapeutically ineffective amount, preferably in an amount of between about 0.007 mg/kg and about 0.3 mg/kg, more preferably between about 0.009 mg/kg and about 0.14 mg/kg, more preferably between about 0.01 mg/kg and about 0.11 mg/kg, more preferably between about 0.014 mg/kg and about 0.07 mg/kg, or wherein said GC is beclomethasone, and wherein said GC is administered in a therapeutically ineffective amount, preferably in an amount of between about 0.001 mg per dosage and about 0.3 mg per dosage, more preferably about 0.05 mg to about 0.2 mg per dosage, even more preferably about 0.1 mg to about 0.15 mg per dosage and/or in a dosage of about 0.001 mg per kg to about 0.005 mg/kg, more preferably about 0.01 mg per kg and about 0.03 mg per kg.

A dose for ginsenosides, preferably Rg1 or Re or a combination thereof, usually varies between about 4 mg and about 400 mg, preferably between about 10 mg and about 300 mg, more preferably between about 20 mg and about 200 mg, more preferably between about 30 mg and about 150 mg, most preferably between about 40 mg and about 70 mg. Preferably, the dose of ginsenosides, preferably Rg1, Re or a combination thereof is between about 0.05 to about 5.5 mg/kg, more preferably between about 0.1 mg/kg to about 4 mg/kg, more preferably between about 0.2 mg/kg to about 3 mg/kg, more preferably between about 0.3 mg/kg and about 2 mg/kg, most preferably between 0.4 and about 1.0 mg/kg. Preferably, said dose for ginsenoside, preferably Rg1, Re or a combination thereof, is at most 5.5 mg/kg, more preferably at most 4 mg/kg, at most 3 mg/kg, at most 2 mg/g, at most 1 mg/kg, more preferably at most 0.5 mg/kg, in particular at most 0.05 mg/kg.

Preferably, a dose for ginsenoside, preferably Rg1, Re or a combination thereof, is between about 1.5 mg/m2 of body surface area and about 250 mg/m2 of body surface area, more preferably between about 5 mg/m2 of body surface area and about 200 mg/m2 of body surface area, preferably between about 10 mg/m2 of body surface area and about 150 mg/m2 of body surface area, between about 15 mg/m2 of body surface area and about 100 mg/m2 of body surface area, most preferably between about 20 mg/m2 of body surface area and about 50 mg/m2 of body surface area.

Said dosage may be provided to a subject in a single dose, or may be provided in multiple doses per day, such as between 2 to 4 doses per day, provided the total amount per day is the same.

Accordingly, the invention preferably relates to a combination of GC and ginsenoside for use according to the invention, wherein said ginsenoside, preferably Rg1 or Re or a combination thereof is administered at a dose of at most about 400 mg/day, preferably at most 200 mg/day, at most 120 mg/day, more preferably at most 70 mg/day, at most 20 mg/day, even more preferably at most 4 mg/day.

Said combination may be administered in any suitable way. For example, said combination may be administered systemically, such as by oral administration, sublingual administration, buccal administration or rectal administration. Alternatively or additionally, said combination may be administered locally, such as transdermal, or via (oral) inhalation or via transnasal administration or via intra-ocular administration. Said combination may also be administered by means of injection, e.g. subcutaneously, intravenously or intramuscularly.

Optionally, said GC may be administered via a different mode of administration compared to said ginsenoside. Preferably, said GC and said ginsenoside are administered via the same mode of administration, preferably transdermal (topically), via inhalation or via oral administration.

Said combination is preferably administered in a formulation with a suitable carrier, preferably an aqueous carrier. In a specific embodiment, said ginsenoside present in said formulation is at least partly incorporated into a micelle, preferably in an outer layer of a micelle, said micelle having a molecular weight of at least 10.000 Da and is preferably administered in a formulation comprising an aqueous medium, such as a hydrogel. Such a combination was particularly effective in achieving an anti-inflammatory effect, whilst preventing the occurrence of side-effects.

Said subject may be any subject suffering from or predisposed of suffering from an inflammatory disease, preferably chronic inflammation. Preferably, said subject is a mammal, more preferably a human.

In principle said inflammatory disease may be any inflammatory disease in which activation of the glucocorticoid receptor may reduce inflammation.

Examples include, but are not limited to, asthma, allergic rhinitis, hay fever, urticaria (hives), atopic eczema, chronic obstructive pulmonary disease (COPD), inflammation of the joints, muscles and tendons, lupus, inflammatory bowel disease, such as Crohn's disease and ulcerative colitis, giant cell arteritis and polymyalgia rheumatica, and multiple sclerosis (MS).

Accordingly, the invention further relates to a combination of GC and a ginsenoside for use in the prevention or treatment of asthma, allergic rhinitis, hay fever, urticaria (hives), atopic eczema, chronic obstructive pulmonary disease (COPD), inflammation of the joints, muscles and tendons, lupus, inflammatory bowel disease, such as Crohn's disease and ulcerative colitis, giant cell arteritis and polymyalgia rheumatica or multiple sclerosis (MS).

The invention further relates to a method of treatment of an inflammatory disease, preferably selected from asthma, allergic rhinitis, hay fever, urticaria (hives), atopic eczema, chronic obstructive pulmonary disease (COPD), inflammation of the joints, muscles and tendons, lupus, inflammatory bowel disease, such as Crohn's disease and ulcerative colitis, giant cell arteritis and polymyalgia rheumatica or multiple sclerosis (MS), comprising administering a combination of GC and a ginsenoside to a subject in need thereof.

The invention further relates to the use of a combination of a GC and a ginsenoside for the preparation of a medicament for the treatment of an inflammatory disease, preferably selected from asthma, allergic rhinitis, hay fever, urticaria (hives), atopic eczema, chronic obstructive pulmonary disease (COPD), inflammation of the joints, muscles and tendons, lupus, inflammatory bowel disease, such as Crohn's disease and ulcerative colitis, giant cell arteritis and polymyalgia rheumatica or multiple sclerosis (MS), comprising administering a combination of GC and a ginsenoside to a subject in need thereof.

In a specific embodiment, the invention preferably relates to a combination of a GC, preferably selected from dexamethasone and beclomethasone, and a ginsenoside, preferably a PPT-type ginsenoside, more preferably selected from Rg1, Re and a combination thereof for use in a method of prevention or treatment of an inflammatory disease, wherein said method comprises administering a GC and a ginsenoside in a molar ratio of between about 1:1 and about 1:2000 to a subject in need thereof.

Said subject in need thereof is typically a human-being in need thereof, preferably an adult human being, such as a human in the age of at least 18 years or more.

### Pharmaceutical Composition

The invention further relates to a pharmaceutical composition comprising a ginsenoside, a pharmaceutically acceptable carrier and preferably a GC, more preferably wherein the molar ratio between said GC and said ginsenoside is preferably between about 1:1 and about 1:2000. Said ginsenoside, GC and said preferred molar ratio are as described herein above.

### Pharmaceutical Carrier

The pharmaceutical composition according to the invention comprises a pharmaceutically acceptable carrier. Said carrier may in principle be any pharmaceutically acceptable carrier known in the art, e.g. a liquid carrier, preferably an aqueous carrier, or a solid carrier such as lactose.

Preferably, said pharmaceutically acceptable carrier is a water-based carrier, more preferably a hydrogel. With such a carrier, a pharmaceutical composition may be formulated as a formulation for topical application, such as a cream or the like. Such a pharmaceutical composition is in particular suitable for dermatological applications, such as eczema.

Alternatively, said pharmaceutical carrier may also be a lipid carrier, or a combination of a liquid carrier and an aqueous carrier.

Preferably, the pharmaceutical composition according to the invention is formulated as a cream, a lotion, a balm, a hydrogel, an ointment, a foam, a gel, a spray, a tablet, a capsule, a lozenge, a topical solution, a topical suspension, an aerosol, an injectable and a syrup, most preferably a hydrogel.

Said pharmaceutical composition according to the invention may further comprise one or more additives, such as a humectant, a stabilizer, a dispersant, a uv-stabilizer, a plasticizer, an emulsifier, an emollient, a preservative or a pH adjusting agent.

In a specific embodiment, the invention relates to a pharmaceutical composition comprising a GC, preferably selected from dexamethasone and betamethasone, a ginsenoside, preferably a PPT-type ginsenoside, preferably selected from Rg1, Re and a combination thereof and a pharmaceutically acceptable carrier, preferably wherein the molar ratio between said GC and said ginsenoside is between about 1:1 and about 1:2000, preferably between about 1:8 and about 1:20. Preferably, herein, said ginsenoside is at least substantially incorporated in a micelle, preferably in an outer layer of a micelle, said micelle having a molecular weight of at least 10.000 Da. Such pharmaceutical composition was found particularly effective in obtaining an anti-inflammatory effect whilst reducing the occurrence of side-effects, in particular side-effects associated with GC-treatment.

The invention further relates to a method for preparing a pharmaceutical composition according to the invention. Said method comprises mixing a ginsenoside, a pharmaceutical carrier and optionally a GC to obtain a pharmaceutical composition according to the invention.

Preferably, said method comprises mixing a ginsenoside formulation according to the invention with a GC, preferably dexamethasone or beclomethasone and a pharmaceutical carrier, preferably a hydrogel to obtain a pharmaceutical composition according to the invention.

Alternatively or additionally, said method comprises mixing a ginsenoside which is at least partly incorporated in an outer layer of a micelle, said micelle having a molecular weight of at least 10.000 Da with a pharmaceutical carrier and preferably a GC. Optionally, said GC may be at least partly incorporated into (an inner cavity of) the micelle and said micelle comprising said GC is mixed with said pharmaceutical carrier to obtain a pharmaceutical composition according to the invention.

The invention further relates to the following clauses.
1. A combination of a glucocorticoid (GC) and a ginsenoside for use in a method of prevention or treatment of an inflammatory disease, wherein said method comprises administering a GC and a ginsenoside in a molar ratio of between about 1:1 and about 1:2000 to a subject in need thereof.
2. The combination for use according to clause 1, wherein said molar ratio is between about 1:4 and about 1:1000, more preferably between about 1:8 and about 1:20.
3. The combination for use according to any one of the preceding clauses, wherein said GC is administered in a therapeutically ineffective amount, preferably at a dose of at most 0.07 mg/kg/day, preferably at most 0.007 mg/kg/day.
4. The combination for use according to any one of the preceding clauses, wherein said ginsenoside is administered in a therapeutic ineffective amount, preferably at a dose of at most 5.5 mg/kg/day, more preferably at most 0.05 mg/kg/day.
5. The combination for use according to any one of the preceding clauses, wherein said GC is selected from the group of beclomethasone and dexamethasone.
6. The combination for use according to any one of the preceding clauses, wherein said inflammatory disease is selected from asthma, allergic rhinitis, hay fever, urticaria, atopic eczema, chronic obstructive pulmonary disease, inflammation of the joints, muscles and tendons, lupus, inflammatory bowel disease, such as Crohn's disease and ulcerative colitis, giant cell arteritis and polymyalgia rheumatica, and multiple sclerosis.
7. The combination for use according to any one of preceding clauses, wherein said ginsenoside is at least partly incorporated into a micelle, preferably at least partly incorporated in an outer layer of a micelle, said micelle having a molecular weight of at least 10.000 Da.
8. The combination for use according to any one of the preceding clauses, wherein said ginsenoside is a PPT-type ginsenoside, preferably selected from Rg1, Re and a combination thereof.
9. The combination for use according to clause 7 or 8 wherein said micelle has a molecular weight of at most 100.000 Da.
10. A ginsenoside formulation comprising a PPT-type ginsenoside at least partly incorporated in a micelle, preferably at least partly incorporated in an outer layer of the micelle, said micelle having a molecular weight of at least 10.000 Da.
11. The ginsenoside formulation according to clause 10, wherein said ginsenoside is a PPT-type ginsenoside, preferably selected from Rg1, Re and a combination thereof.
12. The ginsenoside formulation according to clause 10 or 11, wherein said micelle has a molecular weight of at most 100.000 Da.
13. The ginsenoside formulation according to any one of clauses 10 to 12, further comprising a GC, preferably wherein said GC is encapsulated in said micelle.
14. A ginsenoside for use in a method of prevention or treatment of a side-effect of GC treatment, said method comprising administering a ginsenoside to a subject that is suffering from, or at risk of suffering from, one or more side-effect(s) of GC treatment.
15. The ginsenoside for use according to clause 14, wherein said side-effect is selected from GC-induced diabetes, GC-induced decreased cortisol levels, GC-induced reduced growth velocity, GC-induced wound healing disorders, GC-induced tissue degeneration, GC-induced osteoporosis, GC-induced hypertension, GC-induced weight gain and GC-induced muscle weakness.
16. The ginsenoside for use according to clause 14 or 15, wherein said subject is administered a dose of said ginsenoside of between about 0.7 and about 1.5 mg/kg/day.
17. A ginsenoside formulation according to any one of clauses 10 to 13 for use according to any one of claims 14 to 16.
18. A pharmaceutical composition comprising a GC, a ginsenoside and a pharmaceutically acceptable carrier, wherein the molar ratio between said GC and said ginsenoside is between about 1:1 and about 1:2000, preferably between about 1:4 and about 1:1000, more preferably between 1:8 and 1:20.
19. The pharmaceutical composition according to clause 18, wherein the ginsenoside is a protopanaxatriol (PPT)-type ginsenoside, preferably selected from Rg1 and Re and a combination thereof.
20. The pharmaceutical composition according to clause 18 or 19, wherein said ginsenoside is at least partly incorporated into a micelle, preferably at least partly incorporated in an outer layer of a micelle, said micelle having a molecular weight of at least 10.000 Da.
21. The pharmaceutical composition according to any one of clauses 18 to 20, wherein said pharmaceutical composition is formulated as a cream, a lotion, a balm, a hydrogel, an ointment, a foam, a gel, a spray, a tablet, a capsule, a lozenge, a topical solution, a topical suspension, an aerosol and a syrup.
22. The pharmaceutical composition according to any one of clauses 18 to 21 for use in a method of prevention or treatment of an inflammatory disease.
23. The pharmaceutical composition for use according to clause 22, wherein said inflammatory disease is selected from asthma, allergic rhinitis, hay fever, urticaria, atopic eczema, chronic obstructive pulmonary disease, inflammation of the joints, muscles and tendons, lupus, inflammatory bowel disease, such as Crohn's disease and ulcerative colitis, giant cell arteritis and polymyalgia rheumatica, and multiple sclerosis.
24. A method for preparing a ginsenoside formulation according to any one of clauses 10 to 13, comprising
(a) providing a ginsenoside extract from a plant or plant part of the genus Panax, preferably Panax ginseng;
(b) contacting said ginsenoside extract with an aqueous solvent mixture to obtain a ginsenoside mixture;
(c) at least partly removing said aqueous solvent mixture from said ginsenoside mixture; and optionally
(d) reconstituting the ginsenoside mixture obtained in step c) in water to obtain a ginsenoside formulation.
25. The method according to clause 24, wherein said aqueous solvent mixture comprises a mixture of water and a non-aqueous solvent, preferably an alcoholic solvent, more preferably selected from methanol, ethanol, propanol and isopropanol.
26. The method according to clause 24 or 25, comprising subjecting said ginsenoside extract to a step of column chromatography using a non-polar resin as a stationary phase, preferably a D101 macroporous resin.
27. The method according to any one of clauses 24 to 26, comprising subjecting said ginsenoside extract to a step of decolorization, preferably using a D941 macroporous resin.
28. A ginsenoside formulation obtainable by a method according to any one of clauses 24 to 27.
29. A ginsenoside formulation according to clause 28, comprising a PPT-type ginsenoside at least partly incorporated in a micelle, preferably at least partly incorporated in an outer layer of the micelle, said micelle having a molecular weight of at least 10.000 Da.
30. The ginsenoside formulation according clause 28 or 29, wherein said ginsenoside is a PPT-type ginsenoside, preferably selected from Rg1, Re and a combination thereof.
31. The ginsenoside formulation according to clause 30, wherein said micelle has a molecular weight of at most 100.000 Da.

For the purpose of clarity and a concise description, features are described herein as part of the same or separate embodiments, however, it will be appreciated that the scope of the invention may include embodiments having combinations of all or some of the features described.

The invention is demonstrated by the following examples.

### EXAMPLES

### Example 1: Co-Treatment of Rg1 and GC

### Material and Methods

### Zebrafish Lines and Maintenance

Zebrafish (Danio rerio) were maintained and handled according to the guidelines from the Zebrafish Model Organism Database (http://zfin.org) and in compliance with the directives of the local animal welfare committee of Leiden University. Zebrafish were exposed to a 14 h light and 10 h dark cycle to maintain circadian rhythmicity. Fertilization was performed by natural spawning at the beginning of the light period. Eggs were collected and raised at 28° C. in egg water (60 μg/mL Instant Ocean sea salts and 0.0025% methylene blue). The following zebrafish lines were used in this study: the double transgenic line Tg(mpx:GFPⁱ¹¹⁴/mpeg1:mCherry^{umsF001}), in which neutrophils and macrophages are fluorescently labeled (green and red, respectively) [14,15], and the Tg(9xGCRE-HSV.UI23:EGFP)^{ia20} reporter line, in which the gene encoding enhanced green fluorescent protein (EGFP) is driven by a GRE-containing promoter [16].

### Chemicals

The chemical compounds beclomethasone, dexamethasone, Rg1, TNF-α, actinomycin-D, and cycloheximide were purchased from Sigma-Aldrich (St. Louis, MO, USA).

### Tail Fin Wounding Assay in Zebrafish Larvae

For the tail fin wounding experiments, 20 larvae at 2 or 3 days post-fertilization (dpf) were utilized for each experimental group. For wounding, larvae were anesthetized in egg water containing 0.02% buffered aminobenzoic acid ethyl ester (tricaine; Sigma-Aldrich). Larvae were placed on Petri dishes coated with 2% agarose under a Leica M165C stereomicroscope (Leica Microsystems, Wetzlar, Germany), and the tails were partly amputated using a 1-mm sapphire blade (World Precision Instruments, Sarasota, FL, USA). For quantification of leukocyte migration, larvae were fixed overnight in 4% paraformaldehyde (PFA) at 4° C. The next day, fixed larvae were washed with Phosphate Buffered Saline (PBS) containing 0.1% Tween 20, and stored at 4° C. until imaging.

In the tail fin wounding experiments, the larvae were treated with vehicle (DMSO 0.01%) diluted in egg water, beclomethasone (or dexamethasone), and/or Rg1 at indicated concentrations for 6 h (2 h pre-wounding and 4 h post-wounding).

Imaging of the Tg(mpx:GFPⁱ¹¹⁴/mpeg1:mCherry^{umsF001}) line larvae was performed utilizing a Leica MZ16FA fluorescence stereomicroscope supported by LAS 3.7 software (Leica Microsystems). The macrophages were detected based on their red (mCherry) fluorescence, and neutrophils based on their green (EGFP) fluorescence. To quantify the number of macrophages and neutrophils recruited to the wounded area, the cells in a defined area of the tail were determined by blinded manual counting.

### Determination of the Gr Transactivation Activity in Zebrafish Larvae

To investigate the Gr transactivation activity in zebrafish larvae, the Tg(9xGCRE-HSV.UI23:EGFP)^{ia20} reporter line was used, which expresses enhanced green fluorescent protein (EGFP) under the control of a promoter containing an array of nine GREs [16]. To study the effect of treatment with vehicle, 10 μM beclomethasone (or dexamethasone), 50 μM Rg1, and 10 μM beclomethasone (or dexamethasone) with 50 μM Rg1, 15 embryos per group (2 dpf) were treated with the indicated compounds for 24 h and fixed for visualization to measure the whole-body fluorescence intensity utilizing a Leica MZ16FA fluorescence stereomicroscope, supported by LAS 3.7 software (Leica Microsystems). The integrated intensity of the EGFP signal in the larvae was determined using ImageJ software.

### Measurement of Larval Body Length and Regeneration of the Tail Fin

To determine the length of regenerated tail fin tissue after wounding or the larval body length, chemical treatment (beclomethasone/dexamethasone (10 μM), or Rg1 (50 μM) or beclomethasone/dexamethasone (10 μM) with Rg1 (50 μM) of the embryos (15 per treatment group) was started at 2 hours post-fertilization (hpf) and continued until 5 dpf. During this period, solutions were refreshed daily. For the regeneration experiments, tail fins were wounded at 2 dpf. For determination of the larval length and the regeneration of the tail fins, larvae were fixed at 5 dpf overnight in 4% PFA at 4° C. and imaged the next day by using a Leica MZ16FA fluorescence stereomicroscope supported by LAS 3.7 software (Leica Microsystems), and the length of newly grown tail tissue or the entire larva was measured using ImageJ software. The newly grown tissue can visually be distinguished from the old tissue, enabling precise measurement of the size of the regenerated tissue.

### Whole-Body Glucose Measurement of Zebrafish Larvae

Zebrafish embryos at 2 hpf received a chemical treatment (vehicle, 10 μM beclomethasone, 50 μM Rg1, or 10 UM beclomethasone with 50 μM Rg1), with a daily refreshment of the solutions, until 5 dpf. For dose response curve (0.1, 1, 5, 10, or 15 UM of beclomethasone were introduced alone or in combination with 50 μM of Rg1. At 5 dpf, the larvae (15 per sample) were collected in an Eppendorf tube, washed in egg water (3 times for 10 min), and placed in egg water for 1 h. Subsequently, 100 μL of ice-cold glucose buffer was added to each sample, and the larvae were homogenized using a BulletBlender® for 3 min at 8,000 rpm. The homogenates were then centrifuged at 4° C. for 8 min at 11,000 rpm and the supernatant was stored at −20° C. Whole-body glucose concentrations were determined using a Glucose Colorimetric Assay kit (Cayman Chemical, Ann Arbor, MI, USA), according to the manufacturer's instructions. In each experiment, three biological replicates were used for each treatment group, and the colorimetric assay was performed using technical duplicates.

### Whole-Body Cortisol Measurement of Zebrafish Larvae

Zebrafish embryos at 2 hpf received a chemical treatment (vehicle, 10 μM beclomethasone, 50 μM Rg1, or 10 μM beclomethasone with 50 μM Rg1), with a daily refreshment of the solutions, until 96 hpf. For dose response curve (0.1, 1, 5, 10, or 15 UM of beclomethasone were introduced alone or in combination with 50 μM of Rg1. Then, the treatments were stopped and larvae were incubated in egg water until 5 dpf to avoid cross-reactivity with the compounds of the cortisol antibody used in the ELISA. Then, the treatments were stopped and replaced with egg water. At 5 dpf, the larvae were collected in an Eppendorf tube (30 larvae per sample) and 100 μl of ice-cold egg water was added. After the excess water had been taken out, the samples were glaciated in ethanol (EtOH)/dry-ice bath. Subsequently, the larvae were homogenized using a BulletBlender® for 3 min at 8,000 rpm. Ethyl acetate was added to the homogenate. The homogenates were then centrifuged at 4° C. for 8 min at 11,000 rpm and the supernatant was collected and vaporized. A volume of 150 μl of 0.2% Bovine serum albumin (Sigma-Aldrich) dissolved in PBS was added to the samples and frozen. Whole-body cortisol levels of the zebrafish larvae were determined by ELISA (Demeditec Diagnostics GmbH, Kiel-Wellsee, Germany), following the manufacturer's instructions. In each experiment, three biological replicates were used for each treatment group, and the ELISA was performed using technical duplicates.

### Competitive Glucocorticoid Receptor Binding Assay

To determine the relative binding affinities of the different compounds for the human GR in vitro, the PolarScreen™ Glucocorticoid Receptor Competitor Assay Kit (ThermoFisher Scientific, Waltham, MA, USA) was used following the manufacturer's instructions. Briefly, test compounds were first dissolved in DMSO to a 10 mM stock concentration and further diluted in GR buffer (100 mM potassium phosphate (pH 7.4), 200 mM Na₂MoO₄, 1 mM EDTA, and 20% DMSO). Serial dilutions of the compounds were transferred to a Corning® black 384-well plate and Fluormone GS red™ was added, followed by GR Full Length (partially purified receptor in storage buffer). The plate was incubated for 4 h at RT in the dark and fluorescence polarization was measured with a CLARIOStar Microplate Reader (BMG Labtech, Ortenberg, Germany). Obtained values were normalized to the assay maximum (no ligand) and minimum control (10 UM dexamethasone), and relative percentages of polarization were determined. Dose-response curves were fitted enabling the calculations of IC50 values for each compound.

### Cell Cultures

HeLa (human cervical cancer) cells were cultured in Dulbecco's Modified Eagle's (DMEM) High Glucose (HG) medium without phenol red supplemented with 10% Fetal Calf Serum (FCS) and 10% Glutamax (Sigma-Aldrich). The cells were maintained at 37° C. and 5% CO₂. At 24 h before adding the treatment, the medium was replaced by DMEM HG with 10% charcoal-inactivated serum and 10% Glutamax (Sigma-Aldrich). The cells were exposed to short- and long-term compound treatments. In the short-term treatment, cells were treated with TNF-α (10 ng/ml, Sigma-Aldrich) and either vehicle (0.01% DMSO), beclomethasone (0.01, 0.1, or 1 μM) or dexamethasone (1 μM), Rg1 (20, 100, or 500 μM), or beclomethasone (0.01 or 1 μM, or dexamethasone (1 μM)) in combination with Rg1 (20, 100, 500 μM) for 6 h. In the long-term treatments, the cells were treated for 24 h with one of the following treatments: vehicle (0.01% DMSO), beclomethasone (0.01, 0.1, or 1 μM) or dexamethasone (1 μM), Rg1 (20, 100, or 500 μM), or beclomethasone (0.01 or 1 μM, or dexamethasone (1 μM)) in combination with Rg1 (20, 100 or 200 μM), with or without actinomycin-D (1 ng/ml) or cycloheximide (5 μg/ml). Subsequently, TNF-α (10 ng/ml) was added along with the compound treatments for 6 h (this last step was not added in the actinomycin-D and cycloheximide experiments).

### Immunocytochemistry

To determine the nuclear translocation of GR, immunocytochemistry on GR in Hela cells was performed. The cells were seeded in Nunc™ Lab-Tek™ II Chamber Slide™ System (ThermoFisher Scientific), and cultured in DMEM HG (without phenol red) supplemented with 10% charcoal inactivated serum and 100 mM Glutamax (Sigma-Aldrich) for 48 h. Subsequently, the cells were treated with increasing doses of beclomethasone or Rg1 for 6 h, and fixed using 4% PFA at 4° C. overnight. After washing cells 3 times with PBS for 5 min, PBS with 0.1% Tween 20 (PBST) for 15 min, and PBST with 3% BSA for 30 min, a primary GR antibody (Glucocorticoid Receptor (D6H2L) XP® Rabbit mAb (Cell Signaling Technology, Danvers, MA, USA) was added, diluted in PBST in with 3% BSA (1:1000), and incubated overnight at 4° C. The next day, samples were washed in PBS for 5 min four times. Then, an Alexa Fluor 488® goat anti-rabbit antibody (Sigma-Aldrich) diluted in PBST with 3% BSA (1:500) was added and incubated at room temperature for 2 h. Then, samples were washed in PBS 4 times for 5 min. The cells were mounted using ProLong™ Diamond Antifade Mountant with DAPI (ThermoFisher Scientific) and imaged on a Leica TCS SP8 confocal microscope using a 40× (1.25 NA) objective, (Leica Microsystems). Experiments were performed 3 times in triplicate for each treatment group. In each experiment, 40 randomly selected cells from each treatment group were analyzed. The relative nuclear translocation of the GR was quantified by determining the integrated fluorescence intensity in the nucleus and the whole cell using Fiji ImageJ v1.53c, and these values were corrected for the mean background fluorescence. The percentage of nuclear translocation was assessed by determining the corrected density in the nucleus relative to the corrected density in the whole cell (in %).

Quantitative PCR (qPCR) Analysis

To determine levels of gene expression in zebrafish larvae, 3 dpf larvae were used in both wounded and non-wounded conditions, and after short- and long-term treatments. Groups of 15 larvae were collected in TRIzol reagent (ThermoFisher) and total RNA was isolated using miRNeasy mini kit (Qiagen, Hilden, Germany). For experiments in HeLa cells, cells were seeded in 6 well plates and after treatment, the cells were removed from the wells using TRIzol reagent and mRNA was isolated using miRNeasy mini kit (Qiagen) according to the manufacturer's instructions. The RNA samples were DNAse-treated utilizing the DNA-free™ DNA Removal Kit (ThermoFisher Scientific). The cDNA synthesis was performed using the iScript cDNA synthesis kit (Bio-Rad Laboratories, Hercules, CA, USA) using 1 μg of RNA per sample. For qPCR, 10 μM of forward primer and 10 μM of reverse primer, 12.5 μl of IQ SYBR Green Supermix (Bio-Rad Laboratories), and 2 μl of cDNA were added to the qPCR reaction mixture. Each mixture had a total volume of 25 μl, which was split as duplicates with a volume of 12.5 μl. The qPCR reactions were performed on a MyiQ-single-color real-time PCR detection system (Bio-Rad Laboratories), with initial denaturation for 3 min, 95° C., and 40 cycles of 15 s at 95.5° C., 15 s at 60° C., and 30 s at 72° C. Cycle threshold values (Ct values, i.e. the cycle numbers at which a threshold value of the fluorescence intensity was reached) were determined for each sample. The gene expression level for each sample was normalized using the expression of ppial (peptidylprolyl isomerase Ab (cyclophilin A)) for zebrafish samples, and 18S rRNA for human cells. The fold change per sample (compared to the respective control group) was calculated using the □□Ct method. In each experiment, three biological replicates were used for each treatment group, and reactions were performed in duplicates. The sequences of the qPCR primers for experiments on zebrafish and HeLa cells are presented in Table S1.

TABLE S1
qPCR primers for zebrafish and humanprimers for zebrafish and human

| | qPCR primers for | | qPCR primers for |
|---|---|---|---|
| | zebrafish | | human |
| NAME | Sequence 5′-3′ | NAME | Sequence 5′-3′ |
| ppail_forward | CATCCACAACCTTCCC | hS18_forward | GATGGGCGGGGGAAAAT |
| | GAACAC | | CTTGTACTGGCGTGGAT |
| | | | TCTGC |
| ppail_reverse | ACACTGAAACACGGA | hS18_reverse | GCGAAGGAGAAGACCAC |
| | GGCAAAG | | GACAT |
| il1b_forward | TGTGTGTTTGGGAATC | hFKBP5_forward | TAGGCTTCCCTGCCTCT |
| | TCCA | | CCAAA |
| il1b_reverse | CTGATAAACCAACCGG | hFKBP5_reverse | CATTGCCTGGATGAAGT |
| | GACA | | TTGACG |
| il8_forward | TGTGTTATTGTTTTCC | hPCK1_forward | GGGTTGGTCTTCACTGA |
| | TGGCATTTC | | AGTCC |
| il8_reverse | GCGACAGCGTGGATC | hPCK1_reverse | CGTGGTGGCCATAGACA |
| | TACAG | | ACA |
| fkbp5_forward | CTGCCAGCACAAGA | hGILZ_forward | CCTCTCTCACAGCATACA |
| | TTCGTGAGC | | TCAGATG |
| fkbp5_reverse | GACCCTGCTTATTC | hGILZ_reverse | CTCCGAGACTTTCGAGG |
| | TGATCGGAAA | | AAATAC |
| gr_forward | AACTGGCAACGGTTCT | hNFKBIA_forward | GCCATTGAAGTTGGTAG |
| | ATCAGCTCA | | CCTTCA |
| gr_reverse | TTCTGGTGAAAGAGCA | hNFKBIA_reverse | CCACAGACCTTCCAGGA |
| | GCGG | | GAATG |
| pck1_forward | CAGGGCGATCTGGCG | hIL1B_forward | GTGCAGTTCAGTGATCG |
| | TCTCT | | TACAGG |
| pck1_reverse | CTGCTGTCGATGAACT | hCXCL8_forward | GAGAGTGATTGAGAGTG |
| | CCCG | | GACCAC |
| nfkbiaa_forward | CTTGGGCTAAAGT | hCXCL8_reverse | CACAACCCTCTGCACCC |
| | AGTCACCG | | AGTTT |
| nfkbiaa_reverse | GATGGCAAGGTGCAG | hGR_forward | GAAAAGCCATCGTCAAA |
| | ATACGTG | | AGGG |
| | | hGR_reverse | TGGAAGCAGTAGGTAAG |
| | | | GAGA |
| | | hMMP9_forward | GCCACTACTGTGCCTTT |
| | | | GAGTC |
| | | hMMP9_reverse | CCCTCAGAGAATCGCCA |
| | | | GTACT |

### Western Blotting

Western blots were conducted to determine GR protein levels in HeLa cells. The cells were seeded as described previously in 6-well plates. The cells were exposed to short- and long-term compound treatments. In the short-term treatment, cells were treated with vehicle (0.01% DMSO), beclomethasone (1 μM), Rg1 (20 μM), or beclomethasone in combination with Rg1 (1 and 20 μM, respectively) for 6 h. In the long-term treatments, the cells were treated for 30 h with one of the following treatments: vehicle, beclomethasone (1 μM), Rg1 (20 μM), or beclomethasone in combination with Rg1 (20 and 1 μM, respectively), with or without cycloheximide (5 μg/ml). Cells were harvested using trypsin (0.25% (v/v) trypsin in PBS, no EDTA), washed twice with PBS, and centrifuged. After centrifuging for 3 min at 3,000 rpm, cell pellets were stored at −80° C. until use. Cells were homogenized in potassium phosphate (KPi lysis buffer; 25 mM K₂HPO₄—KH₂PO₄ (pH 6.5)+0.1% (v/v) Triton X-100 (Merck, Darmstadt, Germany)) by sonication (20% amplitude, 3 s on, 3 s off for 4 cycles), using a Vibra-Cell™ VCX 130 sonicator (Sonics, Newtown, CT, USA), while on ice. The total protein concentration of the homogenates was determined using the Quickstart Bradford protein assay (Bio-Rad Laboratories) and measured using an EMax® plus microplate reader (Molecular Devices, Sunnyvale, CA, USA). HeLa cell homogenates (30 μg protein in 10 μl) were added to 30 μl Laemmli sample buffer, vortexed, and, boiled for 5 min at 98° C. Of each sample, 15 μl was loaded in duplicate on a 12% gradient precast SDS-PAGE gel (Bio-Rad Laboratories), and run for approximately 2 h at 90 V (no duplicates were used in the cycloheximide experiment). The experiments were performed 4 times (6 times for the cycloheximide experiment). After running the gel, proteins were transferred to a nitrocellulose membrane (Thermo Fisher Scientific) by using Bio-Rad Power Pac Basic Mini Electrophoresis System at 100 V for 1 h. The blot was washed in a blocking buffer (Tris-buffered saline containing 0.1% Tween-20 (TBST) with 3% BSA) 3 times for 15 min at room temperature. A primary GR antibody (Glucocorticoid Receptor Recombinant Rabbit Monoclonal Antibody (2D8, ThermoFisher Scientific)) at a 1:500 dilution in TBST with 3% BSA was added to the blots and incubated overnight at 4° C. The next day, the blot was washed with TBST 3 times for 5 min. Then, an HRP-conjugated anti-rabbit secondary antibody (Sigma-Aldrich) at 1:2000 dilution in TBST with 3% BSA was added and incubated at room temperature for 2 h. The blot was washed 3 times for 5 min in TBST with 3% BSA and twice for 5 min with TBST. The blot was incubated with 10 ml of Pierce™ enhanced chemiluminescence (ECL) western blotting substrate (ThermoFisher Scientific) for 1-2 min. Chemiluminescence was detected using a ChemiDoc MP imager (Bio-Rad Laboratories) at an exposure time of 1 min. The blot was washed 3 times for 5 min in TBST, then washed for 1 h in TBST with 3% BSA. A β-actin antibody (MA1-91399, ThermoFisher Scientific) was added at a 1:2000 dilution in TBST with 3% BSA to the blot and incubated overnight at 4° C. The next day, the blot was washed 3 times for 5 min in TBST with 3% BSA. Then, an HRP-conjugated anti-mouse secondary antibody (Sigma-Aldrich) at a 1:2000 dilution in TBST with 3% BSA was added and incubated at room temperature for 2 h. Subsequently, the blot was washed 3 times for 5 min in TBST. The blot was incubated with 10 ml of ECL substrate for 1-2 min, and chemiluminescence was visualized. The intensities of the GR and β-actin bands were quantified using ImageJ software, and the GR level was normalized to the level of β-actin.

### Statistical Analysis

Statistical analysis of the experiments was performed using GraphPad Prism software by performing one- or two-way ANOVA with Tukey's post hoc test. The statistics of qPCR data were done on log 2-transformed data. Significance was accepted at P<0.05 and different significance levels are indicated in the graphs: *P<0.05; **P<0.01; and ***P<0.001 for treatments compared to the corresponding vehicle group. #P<0.05; ##P<0.01; and ###P<0.001 for combination treatment compared to beclomethasone or dexamethasone alone. +P<0.05; ++P<0.01; and +++P<0.001 for long-term treatment compared to the corresponding short-term treatment.

### Results

Rg1 has a Synergistic Anti-Inflammatory Effect when Administered as GC Co-Treatment

To study the co-treatment of Rg1 and beclomethasone, we first determined the anti-inflammatory effects of beclomethasone, Rg1, and the combination of these compounds on the migration of neutrophils in the zebrafish tail wounding assay, at 4 h after wounding. The chemical structures of the compounds are shown in FIG. 1A, and a schematic overview of the experiment is presented in FIG. 1B. Beclomethasone was administered at concentrations of 1, 5, 10, 20, and 40 μM, and Rg1 at 10, 20, 40, 80, and 160 μM. The results showed that treatment with lower than 10 μM beclomethasone did not affect the migration of neutrophils towards the wounded site, but that the 20 μM and 40 μM concentrations did significantly reduce the neutrophil migration (FIG. 1C). For Rg1, administration of 40 μM and lower did not alter the neutrophil migration, whereas 80 μM and 160 μM did (FIG. 1C). For the co-treatment, 10 μM beclomethasone was combined with different concentrations of Rg1 (10, 20, 40, and 80 μM). All groups treated with beclomethasone in combination with Rg1 showed decreased neutrophil migration towards the damaged site, compared to the vehicle-treated control groups, and increasing doses of Rg1 resulted in larger inhibition (FIG. 1C). Apparently, co-treatment with Rg1 has a synergistic effect on the anti-inflammatory effect of beclomethasone. Further, the % inhibition of migration of neutrophils to the wounded site have been calculated for beclomethasone at concentration of 10 μM, Rg1 at concentration of 80 μM and a combination of 10 μM beclomethasone and 80 μM of Rg1. The results are shown in FIG. 13, and clearly support the finding that beclomethasone and Rg1 act synergistically together.

Based on these observations, we further examined the anti-inflammatory effect of the combination of 10 μM beclomethasone with 50 μM Rg1. This treatment significantly reduced neutrophil migration, but left the migration of macrophages unaffected (FIGS. 1D, E, S1A), as previously shown in this assay upon treatment with various GCs [17][18]. Moreover, we investigated the effects of Rg1 in combination with dexamethasone, another commonly used synthetic GC. As observed for beclomethasone, the data showed that 10 UM dexamethasone did not affect neutrophil migration, but co-treatment with 50 μM Rg1 caused a significant inhibition of the neutrophil migration to the wounded site (FIGS. S1B, C). This result indicated that the additive anti-inflammatory effect of Rg1 is not only observed upon co-treatment with beclomethasone but can be observed with other GR agonists as well.

To explain the observed effects and translate the results obtained in our zebrafish model to the human situation, we first studied the binding of beclomethasone and Rg1 to the human GR in vitro using a competitive ligand binding assay. The results showed that beclomethasone had a high relative affinity binding to GR, as shown by the IC50 value of 4.9 nM (FIG. 2A). Rg1 had a dramatically (~4,000 times) lower relative binding affinity than the beclomethasone, reflected by an IC50 of 22 μM (FIG. 2A). Using immunocytochemistry on HeLa cell cultures, the translocation of the GR to the nucleus was determined after 6 h of administration of different concentrations of beclomethasone (0.01, 0. 1, and 1 μM), and Rg1 (5, 10, and 20 μM). The concentration of 0.01 μM beclomethasone significantly increased the translocation level compared to vehicle, and maximal nuclear translocation was observed after treatment with 0.1 and 1 μM beclomethasone (FIGS. 2B, C). Rg1 only increased the translocation level compared to the vehicle treatment at 20 μM, still inducing only partial translocation (FIGS. 2B, C).

To study the anti-inflammatory effects of Rg1 in combination with beclomethasone in human cells, we determined the anti-inflammatory effects of beclomethasone, Rg1, and the combination of these compounds on the pro-inflammatory genes IL1B, and IL8 after short-term treatment (6 h). For this purpose, beclomethasone was administered at a concentration of 0.01 μM and Rg1 at 20 μM, which are both concentrations that did not trigger maximal translocation of GR. HeLa cells were treated with TNF-α, which induced the expression of IL1B, and IL8. This induction was slightly suppressed by 0.01 μM beclomethasone and 20 μM Rg1 as individual treatments (FIG. 2D). Interestingly, Rg1 (20 μM) in combination with beclomethasone (0.01 μM) strongly suppressed the induction of the IL1B, and IL8 after (6 h) treatment significantly (FIG. 2D).

To confirm the anti-inflammatory effects of Rg1 in combination with GCs, we determined the mRNA levels of two inflammation-related genes, il1b and il6 by qPCR at 4 h after wounding (FIG. 1F). The cytokine-encoding genes il1b and il6 have previously been shown to be transrepressed in this assay upon treatment with beclomethasone or Rg1 [17]. The results showed that the cytokine-encoding genes il1b and il6 were upregulated after wounding, and that both 10 μM beclomethasone and 50 μM Rg1 as individual treatments slightly decreased the expression of these genes (FIG. 1F). Interestingly, the combination treatment further reduced the expression of il1b and il6 significantly (FIG. 1F).

### Ginsenoside Rg1 Antagonizes GC-Induced Side Effects in Zebrafish

In zebrafish larvae, different biomarkers can be studied as readouts to model side effects of GCs: tissue regeneration [17], larval length, glucose and cortisol levels [19], and the expression of several Gr target genes. In the present study, we have used these biomarkers to examine whether co-treatment with Rg1 could reduce the side effects induced by GCs.

First, a tail fin regeneration assay was performed, in which tail fins are amputated (similarly to the wounding assay) at 2 dpf, and the length of the regenerated fin tissue is determined at 5 dpf. The zebrafish were incubated with compound treatments from 0 to 5 dpf. Our data showed that the vehicle-treated larvae fully regenerated their amputated tail, that beclomethasone (10 μM) inhibited the regeneration, and that Rg1 (50 μM) did not affect the regenerative process, as previously shown [17]. Interestingly, the Rg1 (50 μM)/beclomethasone (10 μM) combination treatment resulted in inhibition of the regeneration, but this inhibition was strongly reduced compared to the beclomethasone treatment (FIGS. 3A, S2A). Thus, our data showed that Rg1 decreased the beclomethasone-induced inhibition of tissue regeneration. To confirm this result using another GC, we studied the effects of Rg1 in combination with dexamethasone (10 μM) in the same assay (FIGS. S2A, B). The results showed that Rg1 also reduced the dexamethasone-induced inhibition of the regenerative process, which suggests that Rg1 generally antagonizes the inhibition of regeneration by GCs.

Second, we studied the effect of the Rg1/beclomethasone combination on the growth of zebrafish. For this purpose, we measured the larval length at 5 dpf after exposing the zebrafish to compound treatments for the entire five-day period (from 0 to 5 dpf). Our data demonstrated that the larval length was significantly decreased when beclomethasone was administered, compared to the effect of vehicle treatment. However, administration of Rg1 did not affect the larval length, and neither did the co-treatment (FIG. 3B), suggesting that Rg1 antagonized the effect of beclomethasone on larval growth.

Third, we studied the effects on whole body glucose and cortisol levels at dpf, which are indicators for GC-induced disruption of the metabolic and endocrine systems, respectively. Larvae were treated over a five-day period (0-5 dpf) for the glucose measurements (assessed using a colorimetric assay) and for four days (0-4 dpf) to determine the effects on cortisol levels (by ELISA). Our results demonstrated that the larvae treated with beclomethasone displayed elevated whole body glucose and reduced whole body cortisol levels compared to the levels of the vehicle-treated larvae (FIGS. 3C, E). In contrast, Rg1 did not affect the whole-body glucose and cortisol concentrations. In the larvae treated with beclomethasone in combination with Rg1, the whole body glucose level was not affected, while the whole body cortisol concentration was only slightly decreased compared to the vehicle-treated larvae (FIGS. 3C, E). These data indicate that Rg1 antagonizes the effects of beclomethasone on the metabolic and endocrine systems.

To determine whether the antagonistic effects of Rg1 on beclomethasone on the glucose and cortisol levels are competitive or non-competitive, dose-response curves were generated for beclomethasone (0.1, 1, 5, 10, and 15 μM) in the absence and presence of Rg1 (50 μM). Our data showed a dose-dependent effect of beclomethasone on the whole-body glucose and cortisol levels. Interestingly, there were significant differences between the groups treated with beclomethasone and the groups treated with the same concentration of beclomethasone in combination with 50 μM Rg1, and the curves of the co-treatment showed a shift to the right compared to the beclomethasone dose-response curve, suggesting that Rg1 acts as a competitive antagonist to beclomethasone (FIGS. 3D, F).

It is generally believed that many of the side effects of GC treatment are a result of the transactivation activity of GR. To study if Rg1 inhibits the transactivation activity of the beclomethasone-activated Gr in zebrafish, the Tg(9xGCRE-HSV.UI23:EGFP)^{ia20} reporter zebrafish line was used. In this line, the EGFP gene expression is driven by a GRE-containing promoter, so the transactivation activity of the Gr can be determined by measuring the EGFP intensity in the larvae.

Larvae at 2 dpf were treated for 24 h, after which they were fixed and visualized under a stereomicroscope to determine the EGFP signal intensity in their bodies, and the relative fluorescence intensity was determined by normalization of the EGFP signal to that of the vehicle group. Our results demonstrated that beclomethasone, as well as dexamethasone significantly increased the relative EGFP intensity (FIGS. 3G, H, S2C). Importantly, Rg1 treatment did not affect the EGFP signal, and in the co-treatment groups, Rg1 abolished the beclomethasone-(and dexamethasone-) induced increase in the EGFP signal (FIGS. 3G, H, S2C). These data indicate that Rg1 antagonizes the GC-induced transactivation activity of Gr in zebrafish.

To further investigate the inhibitory effects of Rg1 on the transactivation activity of the beclomethasone-activated Gr, the expression levels of two endogenous Gr target genes, pck1, and fkbp5, were determined by qPCR at 3 dpf after 6 h term treatment in wounded zebrafish larvae. Our data showed that, while beclomethasone upregulated the expression of pck1 and fkbp5, Rg1 did not affect the expression of these genes (FIG. 3I). Interestingly, the beclomethasone/Rg1 co-treatment did not affect the expression of either of the genes (FIG. 3I). These data confirm the antagonistic effect of Rg1 on the transactivation activity of Gr in zebrafish.

GC Sensitivity is not Reduced after Long-Term Beclomethasone and Rg1 Co-Treatment

We studied if treatment with beclomethasone for a longer time can cause GC resistance in the zebrafish model. To do this, we studied the effects of beclomethasone on the endogenous Gr target genes fkbp5, pck1, and nfkbiaa at 5dpf after short-term (6 h) and long-term (5 days, from 0 to 5 dpf) treatment. Our data showed that beclomethasone (10 μM) upregulated the expression of fkbp5 (FIG. 4A), pck1 (FIG. 4B), and nfkbiaa (FIG. 4C) after short-term treatment, but this upregulation of pck1 and nfkbiaa was significantly reduced after long-term treatment (FIGS. 4A, B), while the upregulation of fkbp5 did not decrease (FIG. 4A). Rg1 (50 μM) did not affect the expression of the genes after either the short- or long-term treatment. Importantly, Rg1 (50 μM) in combination with beclomethasone (10 μM) upregulated the expression of those genes similarly after short- and long-term treatment, indicating that the larvae do not lose GC sensitivity during the long-term combination treatment (FIG. 4A-C).

To investigate the effects of beclomethasone and Rg1 treatment on the expression levels of gr in the zebrafish larvae, a qPCR analysis was performed after short- and long-term treatment at 5 dpf (FIG. 4D). The results of this experiment demonstrated that beclomethasone suppressed the gr mRNA level only after long-term treatment. Rg1 did not affect the gr mRNA level, but did reduce the inhibitory effect of beclomethasone on gr expression after long-term co-treatment. In fact, the co-treatment groups did not show an inhibitory effect on the gr mRNA concentration (FIG. 4D). These data indicate that Rg1 can reduce the beclomethasone-induced suppression of the gr expression, which may explain why the GC sensitivity is not reduced after the Rg1/beclomethasone co-treatment.

To study the effects of Rg1 on the beclomethasone-induced reduction in GR sensitivity in human cells, we first determined the effects of beclomethasone, Rg1, and the combination of these compounds on the pro-inflammatory gene IL1B after short-(6 h) and long-term (30 h) treatment in HeLa cells. For this purpose, beclomethasone was administered at concentrations of 0.01, 0.1, and 1 μM and Rg1 at 20, 100, and 500 μM. Cells were co-treated with TNF-α for the final 6 h. This TNF-α treatment increased the expression of IL1B, and this increase was strongly suppressed by 0.1 and 1 μM beclomethasone after short-term treatment. However, after long-term treatment a significant reduction in the suppressive effects of these high doses of beclomethasone was observed (FIG. 5A). All three doses of Rg1 caused a decrease in IL1B expression after short-term treatment, which was not significantly different after long-term treatment. Similarly, Rg1 in combination with beclomethasone inhibited the induction of the IL1B expression after both short- and long-term treatment, indicating that no loss of GC sensitivity occurs during the long-term combination treatment. Similar data were obtained for Rg1 (20 μM) in combination with dexamethasone (1 μM) on the expression of IL1B (FIG. S4C).

To investigate the effect of Rg1 on the reduced GC sensitivity after long-term treatment in more detail, the effect of two different concentrations of beclomethasone (0.01 and 1 μM) in combination with Rg1 (20 μM) was studied on the expression of IL1B at several time points during short- and long-term treatment in combination with TNF-α treatment. The results demonstrated that the high dose of beclomethasone (1 μM) exhibited a strong suppressive effect on the IL1B expression after short-term treatment, whereas the low concentration (0.01 μM) hardly showed an effect. The inhibitory effect of the high dose of beclomethasone was relatively stable over the 6 h of treatment, and Rg1 had a minor additive effect when it was co-administered (FIG. S4A). However, after long-term treatment, the high dose of beclomethasone (1 μM) did not show a significant suppressive effect on the expression of IL1B, whereas the low dose (0.01 μM) did (FIG. S4B). Interestingly, Rg1 co-treatment with the high dose strongly suppressed the IL1B. These data indicated that the GC sensitivity of HeLa cells which is reduced after a long-term and high-dose treatment with beclomethasone can be prevented by Rg1 co-treatment.

Subsequently, we investigated the effects of Rg1 (20 μM), beclomethasone (1 μM), and Rg1 in combination with beclomethasone on the expression of the pro-inflammatory genes MMP9 and IL8 after short- and long-term treatments. The results showed that the suppressive effects of beclomethasone were significantly reduced after long-term treatment. In contrast, Rg1 slightly suppressed the expression of MMP9, and IL8 after short-term treatment, and more strongly after long-term treatment (FIGS. 5B, C). Interestingly, Rg1 in combination with beclomethasone strongly decreased the expression of both genes after both short- and long-term treatment (FIGS. 5B, C). These data confirm that Rg1 co-treatment prevents the reduced GC sensitivity that is observed after long-term GC treatment.

In addition to these effects on the transrepression activity of GR in HeLa cells, we studied the effects of beclomethasone (1 μM) and Rg1 (50 μM) on the transactivation activity of GR. For this purpose, we measured the expression of the GR target genes FKBP5, NFKBIA, GILZ and SGK1 after short- and long-term treatment. Beclomethasone strongly increased the expression of these genes after short-term treatment (FIG. 5D-G). The beclomethasone-induced expression level of FKBP5 was increased over time (i.e., higher after long-term treatment compared to short-term treatment), while the beclomethasone-induced expression levels of NFKBIA, GILZ, and SGK1 were reduced over time. Rg1 alone did not affect the expression of these genes after either short- or long-term treatment. After short-term co-treatment, Rg1 had an antagonistic effect on the transactivation activity, reducing the beclomethasone-induced expression of the studied genes, while after long-term co-treatment Rg1 abolished the beclomethasone-induced increase in FKBP5 expression, slightly increased the transactivation activities of NFKBIA and GILZ, and did not significantly alter the expression of SGK (FIG. 5D-G). Similar data were observed for the expression of NFKBIA for the dexamethasone (1 μM)/Rg1 (20 μM) combination (FIG. S4D). These data show that the reduced sensitivity after long-term GC treatment is not common to all genes that are transactivated by GR, but for genes that display this reduced sensitivity, it may be prevented by co-treatment with Rg1.

### Rg1 Inhibits the Beclomethasone-Induced Homologous Downregulation of GR

To explain the observed changes in GC sensitivity and confirm the observed effect on the zebrafish gr expression in human cells, the GR mRNA and protein levels were assessed in HeLa cells by qPCR and western blot analysis after short-(6 h) and long-term (24 h) treatment with beclomethasone (1 μM) and Rg1 (20 μM). Our data showed that beclomethasone inhibited the expression of GR at both the mRNA and protein level, and that this inhibitory effect increased over time, whereas Rg1 did not affect the GR mRNA or GR protein level (FIGS. 6A, B, S5A, B). Interestingly, the Rg1/beclomethasone combination treatment did not affect the GR mRNA concentration after either the short- or long-term treatment compared to the vehicle treatment (FIG. 6A), induced only a slight reduction of the GR protein level in the short-term treatment, and did not affect the protein level in the long-term treatment (FIGS. 6B, S5A, B). These data indicated that beclomethasone decreases the GR mRNA and GR protein levels, and that Rg1 does not affect these levels, but that co-treatment with Rg1 can inhibit the beclomethasone-induced reduction in GR expression.

To discriminate between effects on transcription and translation versus effects on mRNA and protein stability, we studied the effect of long-term beclomethasone/Rg1 co-treatment in the presence of the transcription inhibitor actinomycin-D (1 ng/ml) or the protein synthesis inhibitor cycloheximide (5 μg/ml). This way, the effects of different treatments on mRNA and protein stability could be determined. Our results showed that, as expected, actinomycin-D and cycloheximide significantly reduced the GR mRNA and protein concentration in HeLa cells treated with vehicle, beclomethasone, Rg1, or Rg1 in combination with beclomethasone, although the effect on the mRNA level in the beclomethasone-treated group did not reach significance (FIGS. 6A, C, S5C). When we compared the mRNA levels of the actinomycin-D-treated groups, we found that beclomethasone decreased the mRNA levels when transcription is blocked, whereas Rg1 and beclomethasone/Rg1 did not alter the GR mRNA levels (FIG. 6A). Apparently, beclomethasone, and not Rg1, decreases the stability of GR mRNA. Similar data were observed for the protein levels in the presence of cycloheximide (FIGS. 6C, S5C), indicating that protein stability is affected in a similar way as the mRNA levels by beclomethasone, and not Rg1. Taken together, these data demonstrate that the effect of beclomethasone on the GR expression level is largely due to a reduced mRNA and protein stability and that this reduction in stability is prevented by co-treatment with Rg1.

### Example 2: Anti-Inflammatory Effect of PPT, F1, Rg1 and Rh1 and their Effects on Glucose and Cortisol Levels, the Larval Length, and Tissue Regeneration Upon Wounding

### Materials and Methods

### Zebrafish Lines and Maintenance

Zebrafish (Danio rerio) were maintained and handled according to the guidelines from the Zebrafish Model Organism Database (http://zfin.org) and in compliance with the directives of the local animal welfare committee of Leiden University. Zebrafish were exposed to a 14 h light and 10 h dark cycle to maintain circadian rhythmicity. Fertilization was performed by natural spawning at the beginning of the light period. Eggs were collected and raised at 28° C. in egg water (60 μg/ml Instant Ocean sea salts and 0.0025% methylene blue). The following zebrafish lines were used in this study: AB/TL wild type, the transgenic lines Tg(mpx:GFPⁱ¹¹⁴/mpeg1:mCherry^{umsF001})[14, 15], and Tg(9xGCRE-HSV.UI23:EGFPia²⁰)[16], and the mutant lines grs357[20].

### Chemicals

The chemical compounds beclomethasone, prednisolone, dexamethasone, glucuronide-dexamethasone (GDex), PPT, Rh1, F1, Rg1, actinomycin-D, and cycloheximide were purchased from Sigma-Aldrich (St. Louis, MO, USA). Glucose—prednisolone (GPdn) was synthesized in our laboratory, as described previously [21]. MZ31 was kindly provided by Prof. Dr. Hans Aerts (Leiden University, The Netherlands).

### Fish Embryo Acute Toxicity Test (FET)

Fish Embryo Acute Toxicity Test (FET) was performed to determine the tolerated doses of the studied compounds. Because of the relatively high costs of the studied ginsenosides, the FET guidelines of the Organization for Economic Cooperation and Development (OECD, No.236 http://dx.doi.org/10.1787/4.978926-(203709-en) [22]) were adapted according to a previously published protocol that has successfully been used for testing nanoparticles [23], [17,21]. A range of three test concentrations (100, 120, and 150 μM) of Rg1, Rh1, and F1 and (25, 50, and 75M) of PPT was used. A range of four test concentrations of Beclomethasone was used (5, 10, 20, and 25 μM). Stock solutions were made in DMSO, and final dilutions in egg water (60 μg/mL Instant Ocean Sea salts and 0.0025% methylene blue), such that the final DMSO concentration were 0.01%. The following controls were used: a negative control (nC, egg water), a solvent control (sC, 0.01% DMSO in egg water), and positive control (pC), 4 mg/L 3,4-dichloroaniline (Sigma-Aldrich, St. Louis, MO, USA) in egg water.

Embryos were collected around at 1.5 hpf and distributed over standard twenty-four well plates (10 embryos per well), with each well containing 2 mL of test solution. The transgenic line Tg(mpx: GFPi114/mpeg1:mCherry-FumsF001) was used for proper comparison with other experiments in this study. Five 24-well plates were prepared, each with 3 wells containing the Rg1, F1, or Rh1 solutions (100, 120, 150 μM), 3 wells containing the PPT (25, 50, and 75 μM), two for nC and sC, and one for pC (on each plate, 7 wells remained empty). All solutions were refreshed daily.

The plates were kept at a temperature between 26 and 27° C. and exposed to a 12 h light and 12 h dark cycle. At 96, and 120 hpf, the average survival rate (in %) for all experimental groups was determined. Additionally, hatching was recorded at 48 and 72 hpf. Survival rates for the studied compound and pC-treated groups from the five individual plates were averaged. At 96 hpf, the hatching rates (≥80% for nC and sC) and the survival rates (≥90% for nC and sC, ≤70% for pC) were within the criteria for test validity.

### Tail Fin Wounding Assay in Zebrafish Larvae

For the tail fin wounding experiments, 2- or 3-days post-fertilization (dpf) larvae were anesthetized in egg water containing 0.02% buffered aminobenzoic acid ethyl ester (tricaine; Sigma-Aldrich). Larvae were placed in Petri dishes coated with 2% agarose under a Leica M165C stereomicroscope (Leica Microsystems, Wetzlar, Germany), and the tails were partly amputated using a 1-mm sapphire blade (World Precision Instruments, Sarasota, FL, USA).

### Quantification of Leukocyte Migration

In experiments in which the leukocyte migration was used as a readout, larvae were subjected to treatment with chemicals as indicated, starting at 2 h before the amputation (pretreatment), and treatment was continued for 4 h after the tail fin amputation (20 larvae per group, unless indicated otherwise). In some experiments, a 24 h treatment with MZ31 (10 μM) was started at 2 dpf, and these treatments were continued during the pretreatment and treatment with the other chemicals. At 4 h after amputation, the larvae were fixed in 4% paraformaldehyde (PFA) overnight at 4° C. The next day, fixed larvae were washed with Phosphate Buffered Saline (PBS) containing 0.1% Tween 20 and stored at 4° C. until imaging. Imaging of larvae from the Tg(mpx: GFPⁱ¹¹⁴/mpeg1:mCherry^{umsF001}) line was performed utilizing a Leica MZ16FA fluorescence stereomicroscope supported by LAS 3.7 software (Leica Microsystems). The macrophages were detected based on red (mCherry) fluorescence and neutrophils on their green (EGFP) fluorescence. To quantify the number of macrophages and neutrophils recruited to the wounded area, the cells in a defined area of the tail were determined by blinded manual counting. When larvae from the grs357 mutant lines were used, neutrophil labeling was performed with the TSA fluorescein detection kit (PerkinElmer) for specific staining of Myeloperoxidase (Mpx)-positive cells, following the manufacturer's instructions.

### Measurement of Larval Body Length and Regeneration of the Tail Fin

In experiments in which the length of regenerated tail fin tissue after wounding, or the larval body length, was used as a readout, chemical treatments were started at 2 hours post-fertilization (hpf) and continued until 5 dpf, as indicated (15 larvae per treatment group). During this period, solutions were refreshed daily. For the regeneration experiments, tail fins were amputated at 2 dpf. At 5 dpf, larvae were fixed overnight in 4% PFA at 4° C. For determination of the larval length and the regeneration of the tail fins, larvae were imaged using a LeicaMZ16FA fluorescence stereomicroscope supported by LAS 3.7 software. The length of the entire larva or the newly grown tissue was measured using ImageJ software. The newly grown tissue can be visually distinguished from the old tissue, enabling precise measurement of its length.

### Whole-Body Glucose Measurement of Zebrafish Larvae

Zebrafish embryos at 2 hpf received a chemical treatment with or without MZ31, with a daily refreshment of the solutions, until 5 dpf. Tail fin wounding were performed at 2 days post-fertilization (dpf). At 5 dpf, the larvae (15 per sample) were placed in egg water for 1 h and collected in an Eppendorf tube, washed in egg water (3 times for 10 min). Subsequently, 100 μL of ice-cold glucose buffer was added to each sample, and the larvae were homogenized using a BulletBlender® for 3 min at 8,000 rpm. The homogenates were then centrifuged at 4° C. for 8 min at 11,000 rpm and the supernatant was stored at −20° C. Whole-body glucose concentrations were determined using a Glucose Colorimetric Assay kit (Cayman Chemical, Ann Arbor, MI, USA), according to the manufacturer's instructions. In each experiment, three biological replicates were used for each treatment group, and the colorimetric assay was performed using technical duplicates.

### Whole-Body Cortisol Measurement of Zebrafish Larvae

Zebrafish embryos at 2 hpf received a chemical treatment with or without MZ31, with a daily refreshment of the solutions, until 96 hpf. Tail fin wounding were performed at 2 days post-fertilization (dpf). At 96hpf, the treatments were stopped, and larvae were incubated in egg water until 5 dpf to avoid cross-reactivity between the compounds and the cortisol antibody used in the ELISA. At dpf, the larvae were collected in an Eppendorf tube (30 larvae per sample), and 100 μl of ice-cold egg water was added. After the excess water had been taken out, the samples were glaciated in ethanol (EtOH)/dry-ice bath. Subsequently, the larvae were homogenized using a BulletBlender® for 3 min at 8,000 rpm. Ethyl acetate was added to the homogenate. The homogenates were then centrifuged at 4° C. for 8 min at 11,000 rpm, and the supernatant was collected and vaporized. A volume of 150 μl of 0.2% bovine serum albumin (Sigma-Aldrich) dissolved in PBS was added to the samples and frozen. Whole-body cortisol levels of the zebrafish larvae were determined using a Cortisol free in Saliva ELISA kit (Demeditec Diagnostics GmbH, Kiel-Wellsee, Germany), following the manufacturer's instructions. In each experiment, three biological replicates were used for each treatment group, and the ELISA was performed using technical duplicates.

### Determination of the Gr Transactivation Activity in Zebrafish Larvae

To investigate the Gr transactivation activity in zebrafish larvae, the Tg(9xGCRE-HSV.UI23:EGFP^{ia20}) reporter line was used, which expresses enhanced green fluorescent protein (EGFP) under the control of a promoter containing an array of nine GREs [16]. To study the effect of chemical treatment, 2 dpf zebrafish embryos (15 per group) were treated with indicated compounds for 24 h and fixed for visualization to measure the whole-body fluorescence intensity utilizing a Leica MZ16FA fluorescence stereomicroscope, supported by LAS 3.7 software (Leica Microsystems). The integrated intensity of the EGFP signal in the larvae was determined using ImageJ software.

### Overexpression of Gba2 in Zebrafish

For overexpression of gba2 in zebrafish, a plasmid containing the cDNA encoding the zebrafish gba2 gene fused to a CMV promoter (pDEST-zeo-zGBA2) [21,23,24] was used. One-cell stage zebrafish embryos were injected with this plasmid, diluted in nuclease-free water (1 nl/egg with a final concentration of 80 pg/egg), using the Automated Microinjection System Version 3 AMS-03 (Life Science Methods BV, Leiden, The Netherlands). After the injection, chemical treatment was performed as indicated.

Quantitative PCR (qPCR) Analysis

To determine mRNA levels of specific genes in zebrafish, 3 dpf larvae were used (wounded and non-wounded) and after 6 h of chemical treatments (2 h pre-wounding, 4 h post-wounding), as indicated. Per sample, groups of 15 larvae were collected in TRIzol reagent (Thermo Fisher Scientific, Waltham, MA, USA), and total RNA was isolated using the miRNeasy mini kit (Qiagen, Hilden, Germany). For similar experiments in Hela cells, cells were seeded in 6 well plates, and after 6 h of chemical treatment, the cells were removed from the wells using TRIzol reagent, and total RNA was isolated using the miRNeasy mini kit according to the manufacturer's instructions. The RNA samples were DNAse-treated utilizing the DNA-free™ DNA Removal Kit (Thermo Fisher Scientific). The cDNA synthesis was performed using the iScript cDNA synthesis kit (Bio-Rad Laboratories, Hercules, CA, USA) using 1 μg of RNA per sample. For the PCR reactions, 10 μM of forward and 10 μM of reverse primer, 12.5 μl of iQ SYBR Green Supermix (Bio-Rad), and 2 μl of cDNA were added to the qPCR reaction mixture. Each mixture had a total volume of 25 μL, which was split as duplicates with a volume of 12.5 μL. The reactions were performed on a MyiQ-single-color real-time PCR detection system (Bio-Rad Laboratories) with initial denaturation for 3 min at 95° C. and 40 cycles of 15 s at 95.5° C., 15 s at 60° C., and 30 s at 72° C. Cycle threshold values (Ct values, i.e. the cycle numbers at which a threshold value of the fluorescence intensity was reached) were determined for each sample. The gene expression level for each sample was normalized using the expression of ppial (peptidylprolyl isomerase Ab (cyclophilin A)) for zebrafish samples, 18S rRNA for human cells. The fold change per sample (compared to the respective control group) was calculated using the ΔΔCt method. In each experiment, three biological replicates were used for each treatment group, and reactions were performed in duplicates. The sequences of the qPCR primers for experiments on zebrafish, and HeLa cells are presented in Table 1 and 2, respectively.

TABLE 1
Zebrafish qPCR primers

| Name | Sequence 5′-3′ |
|---|---|
| ppail_forward | CATCCACAACCTTCCCGAACAC |
| ppail_reverse | ACACTGAAACACGGAGGCAAAG |
| illb_forward | TGTGTGTTTGGGAATCTCCA |
| illb_reverse | CTGATAAACCAACCGGGACA |
| il6_forward | CGCTAAGGCAACTGGAAGAC |
| il6_reverse | CCAGACCACTGGGAAACACT |
| il8_forward | TGTGTTATTGTTTTCCTGGCATTTC |
| il8_reverse | GCGACAGCGTGGATCTACAG |
| mmp9_forward | CATTAAAGATGCCCTGATGTATCCC |
| mmp9_reverse | AGTGGTGGTCCGTGGTTGAG |
| mmp13a_forward | ATGGTGCAAGGCTATCCCAAGAGT |
| mmp13a_reverse | GCCTGTTGTTGGAGCCAAACTCAA |
| fkbp5_forward | TCTGCCAGCACAAGATTCGTGAGC |
| fkbp5_reverse | GACCCTGCTTATTCTGATCGGAAA |
| pck1_forward | CAGGGCGATCTGGCGTCTCT |
| pckl_reverse | CTGCTGTCGATGAACTCCCG |
| nfkbiaa_forward | CTTGGGCTAAAGTAGTCACCG |
| nfkbiaa_reverse | GATGGCAAGGTGCAGATACGTG |

TABLE 2
Human qPCR primers

| Name | Sequence 5′-3′ |
|---|---|
| S18_forward | CATGGGGGGGGGANAAT |
| S18_reverse | CTTGTACTGGCGTGGATTCTGC |
| FKBP5_forward | GCGAAGGAGAAGACCACGACAT |
| FKBP5_reverse | TAGGCTTCCCTGCCTCTCCAAA |
| PCK1_forward | CATTGCCTGGATGAAGTTTGACG |
| PCK1_reverse | GGGTTGGTCTTCACTGAAGTCC |
| GILZ_forward | CGTGGTGGCCATAGACAACA |
| GILZ_reverse | CCTCTCTCACAGCATACATCAGATG |
| NFKBLA_forward | CTCCGAGACTTTCGAGGAAATAC |
| NFKBIA_reverse | GCCATTGAAGTTGGTAGCCTTCA |
| ILIB_forward | CCACAGACCTTCCAGGAGAATG |
| ILIB_revese | GTGCAGTTCAGTGATCGTACAGG |
| CXCL8_forward | GAGAGTGATTGAGAGTGGACCAC |
| CXCL8_reverse | CACAACCCTCTGCACCCAGTTT |
| MMP13_forward | CCTTGATGCCATTACCAGTCTCC |
| MMP13_reverse | AAACAGCTCCGCATCAACCTGC |
| GR-forward | CTTGTACTGGCGTGGATTCTGC |
| GR-reverse | TGGAAGCAGTAGGTAAGGAGA |

### Cell Culture and Transfection

HeLa (human cervical cancer) cells, purchased from (ATCC), were cultured in Dulbecco's Modified Eagle's (DMEM) High Glucose (HG) medium without Phenol Red, supplemented with 10% Fetal Calf Serum (FCS) and 10% Glutamax (Sigma-Aldrich). The cells were maintained at 37° C. and 5% CO₂. At 24 hours before treatment, cells were seeded in 6 well plates and allowed to adhere in the presence or absence of MZ31 (1 μM). MZ31 was added overnight. After adherence and reaching 80% confluence, cells were treated with indicated chemicals for 6 h, in the presence or the absence of TNF-α (10 ng/ml) (Sigma-Aldrich) and/or MZ31 (1 μM).

For overexpression of GBA2 in HeLa cells, a plasmid containing the cDNA encoding the human GBA2 gene fused to a CMV promoter (pDEST-zeo-hGBA2)[21,23,24] was transfected into ~70% confluent HeLa cells. This plasmid was mixed with FuGENE HD transfection reagent (Promega, Madison, WI, USA) and 500 μl of serum-free DMEM. The mixture was incubated for 20 min at room temperature and added to the HeLa cells cultured in supplemented DMEM. After 2 days, the medium was changed, and chemical treatments were performed, as indicated, for 6 h.

### Statistical Analysis

Statistical analysis of the experiments was performed using GraphPad Prism software by performing one- or two-way ANOVA with Tukey's post hoc test.

### The Anti-Inflammatory Action of Monoglycosylated Ginsenosides Depends on Glucocorticoid Receptor Function, and not on Deglycosylation by Glucosylceramidase Beta 2

To study whether monoglycosylated ginsenosides, such as F1 and Rh1, have similar effects and dependency on Gr and/or Gba2, we studied their anti-inflammatory action in zebrafish larvae. To investigate the structure-function relationship of these compounds, we used the ginsenosides Protopanaxatriol (PPT), F1, Rh1 and Rg1, and the glucocorticoid drug beclomethasone (Bec) as a positive control (compounds structures are shown in FIG. 7A). PPT is not glycosylated, and F1 and Rh1 consist of the aglycone PPT with one glucose group at the C-20 or the C-6 position of the PPT backbone structure, respectively. Rg1 consists of the aglycone PPT with one glucose group attached at the C-6 and one glucose group at the C-20 position. First, to determine the appropriate doses of the studied ginsenosides in this study, a Fish Embryo Acute Toxicity Test (FET) was performed in which zebrafish embryos were exposed to a concentration range of these compounds between 0 and 120 hpf, and the effects on hatching and survival were determined (FIGS. S7A, B). For PPT, no effects were observed until 50 μM, whereas considerable effects were shown at 75 μM. For F1, Rh1, and Rg1, no effects were observed until 100 μM, and minimal effects were observed at 120 μM. Based on these results, we selected doses of 50 μM for PPT, and 100 μM for F1, Rh1, and Rg1.

Using these doses, we determined the anti-inflammatory effect of these compounds in zebrafish larvae. We induced a local inflammation in zebrafish larvae at 3 days post fertilization (dpf) by wounding the tail fin (a schematic drawing of the experiment is shown in FIG. 7B). Our results showed that treatment of the larvae with the ginsenosides PPT, F1, Rh1, and Rg1, like Bec treatment, inhibited the migration of neutrophils without affecting the macrophage migration (FIG. 7C). For all compounds, the inhibition of neutrophil migration was abolished in zebrafish larvae from a mutant line with a deficiency in Gr function (FIG. S7C), indicating that the anti-inflammatory effects of studied ginsenosides are mediated by Gr.

Further, we investigated whether the anti-inflammatory effects of monoglycosylated ginsenosides also required the action of Gba2 using the specific GBA2 inhibitor MZ31. Treatment with MZ31 did not affect the reduction in neutrophil migration by the monoglycosylated ginsenosides F1 and Rh1, or the aglycone ginsenoside PPT. In contrast, this inhibitor abolished the effect of the polyglycosylated ginsenoside Rg1 (FIG. 7D). Taken together, these data indicate that the anti-inflammatory effects of monoglycosylated ginsenosides F1 and Rh1 are independent of Gba2, suggesting that addition of a single glucose group at either the C-6 or C-20 position of PPT does not interfere with its activation of Gr, whereas the presence of glucose groups at both positions requires deglycosylation by Gba2 before it can activate Gr.

To further study the anti-inflammatory effects of the ginsenosides, we studied their effects on the expression of genes encoding pro-inflammatory proteins. Our results demonstrated that there is selectivity in the suppression of inflammation-induced gene expression by ginsenosides. All studied ginsenosides effectively suppressed the expression levels of il1b, il6 and mmp9, similarly to Bec (FIG. 7E). However, ginsenosides do not suppress the expression of il8 and mmp13 as effectively as Bec. Only a minor effect of PPT and F1 on the expression of these two genes was found, and no effect of Rh1 and Rg1 was observed (FIG. 7E). The characteristic that distinguishes Rh1 and Rg1 from PPT and F1 is the glucose group at the C-6 position, so based on these data we suggest that the glucose at the C-6 position in Rh1 and Rg1 is not cleaved off by Gba2, whereas this enzyme effectively removes the glucose group from C-20 in F1. As a result, PPT and F1 have a similar effect, as well as Rh1 and Rg1 on the il8 and mmp13 expression.

### Glycosylation of Ginsenosides Causes a Significant Reduction in Side Effects

To evaluate the side effects of the monoglycosylated ginsenosides, zebrafish embryos were treated with Bec, PPT, F1, Rh1, and Rg1, and effects were monitored on the glucose and cortisol levels, the larval length, and tissue regeneration upon wounding. Treatment with Bec, used as a positive control, induced an increase in the glucose level (FIG. 8A), a decrease in cortisol level (FIG. 8C), a decrease in larval length (FIG. 8E), and inhibited the tissue regeneration. (FIGS. 8F, S8A). The aglyconic ginsenoside PPT did not affect the glucose level (FIG. 8A) but did decrease the cortisol concentration (FIG. 8C), showed a minor effect on larval length (FIG. 8E), and considerably inhibited the regeneration of the tail fin after wounding (FIGS. 8F, S8A). Interestingly, the glycosylated ginsenosides F1, Rh1, and Rg1 did not show any side effects, either on the glucose level (FIG. 8A), the cortisol concentration (FIG. 8C), the larval length (FIG. 8E), or the tissue regeneration (FIGS. 8F, S8A). Thus, we conclude that glycosylation of PPT abolishes all its side effects.

Interestingly, upon wounding, F1 did not affect the glucose level (FIG. 8B) but did show a minor effect on the cortisol level and on tissue regeneration (FIGS. 8D, F, S8A). These latter effects were Gba2-dependent, which was demonstrated using the Gba2 inhibitor MZ31 (FIGS. 8D, F), suggesting that upon wounding F1 is deglycosylated due to increased activity of Gba2. In contrast, Rh1 and Rg1 did, upon wounding, still not show any effect on the glucose or cortisol level or on tissue regeneration (FIGS. 8B, D, F, S8A). These results indicated that Gba2 could cleave off the glucose groups at C20 in ginsenoside F1 (thereby converting it to PPT), but not at C6 in Rh1 and Rg1, leaving the latter two ginsenosides glycosylated at this position. As a result, glycosylation at the C-6 position (as in Rh1) abolished the side effects of PPT also after wounding, but glycosylation at the C-20 position (as in F1) failed to abolish the side effects of PPT, probably because the glucoses are cleaved off due to the increased local Gba2 activity upon wounding.

To study whether ginsenosides PPT, F1, and Rh1 can trigger GR transactivation activities, we used a reporter fish line in which the GFP gene is fused to a GRE-containing promoter Tg(9xGCRE-HSV.UI23:EGFP)^{ia20}. Treatment of larvae from this line with beclomethasone resulted in a significant increase in the GFP signal throughout the body of the larvae, whereas none of the ginsenosides PPT, F1, Rh1, and Rg1 altered the fluorescence intensity (FIGS. 8G, H). Moreover, those ginsenosides did not increase the expression of fkbp5, pck1, and nfkbiaa, which are well-known target genes of the transactivation activity of the Gr (FIGS. 8I, S8B). These data indicate that it is a general characteristic of ginsenosides that they do not induce the transactivation activity of the Gr.

### Example 3: Effect of Ginsenoside Re in Zebrafish

In addition to Rg1, the effect of addition of ginsenoside Re in zebrafish was tested on anti-inflammatory effects and putative side effects as described in Example 1. It was shown that Re inhibited the migration of neutrophils to the wounded tail area and did not inhibit the migration of macrophages (FIGS. 9A, B). In contrast to glucocorticosteroids no side effects could be observed after addition of Re with respect to whole body glucose and whole body cortisol (FIGS. 9C, D). In addition Re did not affect regenerative tissue (FIG. 9E).

### Example 4: Preparation of Micelles from Ginseng Leaves

Dried leaves of the plant Panax ginseng (8 gram) were boiled in water (350 mL), filtrated and loaded on a 20 mL Bed Volume (BV) column filled with macroporous resin (type D101). After extensively washing with water (15 BV's) the ginsenosides were eluted with 60% EtOH. The ginsenoside solution was decolorized by passing through a 20 mL BV column filled with type D941 macroporous resin. Subsequently, the solvents were slowly evaporated, after at 80° C. under reduced pressure (375 hPa) using a glass vials and a evaporator (P12-Multivapor, Buchi). After evaporation of both the ethanol and the water, a transparent film was formed on the wall of the glass vial. The film was solved in H₂O resulting in a clear solution (see FIG. 12A) containing about 15 mg/mL ginsenosides.

In parallel 100 mg pure Re was solved in 10 mL of ethanol. This resulted in a clear solution (see FIG. 12A). The ethanol was slowly removed at 80° C. under low pressure (375 hPa) using a glass vials and a evaporator (P12-Multivapor). Again a clear film was formed on the wall of the glass tubes. The film was solved in 10 mL of H₂O resulting a cloudy solution (see FIG. 12A). Microscopic determination revealed crystallization of Re (FIG. 12B) and no micelles were obtained.

Micelle formation of ginsenoside extract obtained from dried leaves as described above was repeated in the presence of 0.6 mg of dexamethasone. Dexamethasone was first dissolved in ethanol in a concentration of 10 mg/mL and 0.6 mg of dexamethasone was added to the ethanolic ginsenoside fraction obtained after purification and decolorization by D101 and D941 resins respectively, using the method described herein above. The content of Re and Rg1 in this ethanolic ginsenoside fraction was 4 mg/10 mL as determined by HPLC (see below). The molecular ratio between dexamethasone and Re/Rel in this solution was around 5:70. The ethanol fraction was slowly removed at 80° C. under reduced pressure (375 hPa) using a glass vials and a evaporator to obtain a film. The film was dissolved in H₂O resulting in a clear aqueous solution. The content and the presence of micelles were assessed by size exclusion chromatography as described below.

The size and content of the micelles were determined by means of size exclusion chromatography. 1 mL of aqueous ginsenoside/dexamethasone solution was loaded on a Sephadex G50 size exclusion column (size 25×1 cm) and eluted under gravity with water. Blue dextran (MW=2.000.000 Da) and erythrosine (880 Da) were used as molecular weight markers. Fractions of 2.0 mL were collected. The fractions were dried and dissolved in 70% methanol and the ginsenoside content was analyzed by means of HPLC. HPLC conditions were:
HPLC equipment: HPLC/DAD Aligent 1200 series, a quaternary pump connected to a DAD and autosampler.
Stationary phase: Kinetex 2.6 μm, C18 100 Å, 100×4.6 mm-Phenomenex, USA
Mobile phase:
   A: 0.03% o-phosphoric acid (H₃PO₄) in Ultrapure H₂O
   B: 0.03% o-phosphoric acid (H₃PO₄) in Acetonitrile (LiChrosolv, Millipore-Merck, Darmstadt, Germany; quality far UV)
Gradient:

| Time (Minutes) | % B |
|---|---|
| 0.00 | 17.0 |
| 12.00 | 22.0 |
| 18.00 | 30.0 |
| 31.00 | 36.0 |
| 33.00 | 60.0 |
| 34.50 | 60.0 |
| 34.51 | 17.0 |
| 36.50 | 17.0 |

Conditions
   Flow: 0.8 ml/min
   Injection volume: 20 μL
   Column temperature: 35° C.
   Detection wavelength: 203 and 241 nm

FIG. 13 shows a size exclusion chromatogram showing the molecular weight and content of the micelles.

The highest concentration of micelles with a high content of ginsenosides Re and Rg1 was eluted after 30 mL (FIG. 13, FIG. 14B). Based on the elution profile of the molecular markers blue dextran (MW 2.000.000 Da; peak elution at 17 mL and erythrosine (MW 880 Da; peak elution at 55 mL) it was estimated that the size of the micelles containing mainly the ginsenosides Re and Rg1 was about 80,000 Da.

These results indicate that the micelles contain around 50 to 100 molecules of mainly Re, Rg1 or both. Interestingly dexamethasone was also found to be co-eluted with the Re and Rg1 containing micelles. In addition dexamethasone, having a molecular weight of 392.5 Da in a single molecular form, was expected to be eluted after erythrosine e.g. in fractions after 55 mL, but dexamethasone could not be detected in these fractions. This indicates that dexamethasone was encapsulated in a micelle formed by Re, Rg1 or both. Further, it is shown that with this method a high concentration of PPT-type ginsenosides, optionally in combination with dexamethasone, in water, could be obtained.

### REFERENCES

1. Homo-Delarche, F.; Fitzpatrick, F.; Christeff, N.; Nunez, E. A.; Bach, J. F.; Dardenne, M. Sex steroids, glucocorticoids, stress and autoimmunity. J. Steroid Biochem. Mol. Biol. 1991, 40, 619-637, doi: 10.1016/0960-0760(91)90285-d.
2. Hall, B. M.; Hodgkinson, S. J.; Quin, J. Corticosteroids in autoimmune diseases. Aust. Prescr. 1999, 22, 9-11, doi:DOI: 10.18773/austprescr.1999.008.
3. Trombetta, A. C.; Meroni, M.; Cutolo, M. Steroids and Autoimmunity. Front. Horm. Res. 2017, 48, 121-132.
4. Ashwell, J. D.; Lu, F. W. M.; Vacchio, M. S. Glucocorticoids in T Cell Development and Function. Annu. Rev. Immunol. 2000, 18, 309-345.
5. Ratman, D.; Vanden Berghe, W.; Dejager, L.; Libert, C.; Tavernier, J.; Beck, I. M.; De Bosscher, K. How glucocorticoid receptors modulate the activity of other transcription factors: a scope beyond tethering. Mol. Cell. Endocrinol. 2013, 380, 41-54, doi: 10.1016/j.mce.2012.12.014.
6. Reichardt, H. M.; Tuckermann, J. P.; Gottlicher, M.; Vujic, M.; Weih, F.; Angel, P.; Herrlich, P.; Schutz, G. Repression of inflammatory responses in the absence of DNA binding by the glucocorticoid receptor. EMBO J. 2001, 20, 7168-7173, doi: 10.1093/emboj/20.24.7168.
7. Patel, G. C.; Millar, J. C.; Clark, A. F. Glucocorticoid Receptor Transactivation Is Required for Glucocorticoid-Induced Ocular Hypertension and Glaucoma. Invest. Ophthalmol. Vis. Sci. 2019, 60, 1967-1978, doi: 10.1167/iovs.18-26383.
8. Tanigawa, K.; Nagase, H.; Ohmori, K.; Tanaka, K.; Miyake, H.; Kiniwa, M.; Ikizawa, K. Species-specific differences in the glucocorticoid receptor transactivation function upon binding with betamethasone-esters. Int. Immunopharmacol. 2002, 2, 941-950, doi: 10.1016/s1567-5769(02)00036-x.
9. Newton, R. Molecular mechanisms of glucocorticoid action: what is important? Thorax 2000, 55, 603-613.
10. Gordijn, M. S.; Gemke, R. J.; van Dalen, E. C.; Rotteveel, J.; Kaspers, G. J. Hypothalamic-pituitary-adrenal (HPA) axis suppression after treatment with glucocorticoid therapy for childhood acute lymphoblastic leukaemia. Cochrane database Syst. Rev. 2012, CD008727, doi: 10.1002/14651858.CD008727.pub2.
11. Straub, R. H.; Cutolo, M. Glucocorticoids and chronic inflammation. Rheumatology (Oxford). 2016, 55, ii6-ii14, doi: 10.1093/rheumatology/kew348.
12. Schaaf, M. J. M.; Cidlowski, J. A. Molecular mechanisms of glucocorticoid action and resistance. J. Steroid Biochem. Mol. Biol. 2002, 83, 37-48, doi: 10.1016/s0960-0760(02)00263-7.
13. Wilkinson, L.; Verhoog, N. J. D.; Louw, A. Disease- and treatment-associated acquired glucocorticoid resistance. Endocr. Connect. 2018, 7, R328-R349, doi: 10.1530/EC-18-0421.
14. Renshaw, S. A.; Loynes, C. A.; Trushell, D. M. I.; Elworthy, S.; Ingham, P. W.; Whyte, M. K. B. A transgenic zebrafish model of neutrophilic inflammation. Blood 2006, 108, 3976-3978, doi: 10.1182/blood-2006-05-024075.
15. Bernut, A.; Herrmann, J.-L.; Kissa, K.; Dubremetz, J.-F.; Gaillard, J.-L.; Lutfalla, G.; Kremer, L. Mycobacterium abscessus cording prevents phagocytosis and promotes abscess formation. Proc. Natl. Acad. Sci. U.S.A 2014, 111, E943-E952, doi: 10.1073/pnas.1321390111.
16 Benato, F.; Colletti, E.; Skobo, T.; Moro, E.; Colombo, L.; Argenton, F.; Dalla Valle, L. A living biosensor model to dynamically trace glucocorticoid transcriptional activity during development and adult life in zebrafish. Mol. Cell. Endocrinol. 2014, 392, 60-72, doi: 10.1016/j.mce.2014.04.015.
17. He, M.; Halima, M.; Xie, Y.; Schaaf, M. J. M.; Meijer, A. H.; Wang, M. Ginsenoside Rg1 Acts as a Selective Glucocorticoid Receptor Agonist with Anti-Inflammatory Action without Affecting Tissue Regeneration in Zebrafish Larvae. Cells 2020, 9, doi: 10.3390/cells9051107.
18. Chatzopoulou, A.; Heijmans, J. P. M.; Burgerhout, E.; Oskam, N.; Spaink, H. P.; Meijer, A. H.; Schaaf, M. J. M. Glucocorticoid-induced attenuation of the inflammatory response in zebrafish. Endocrinology 2016, 157, 2772-2784, doi: 10.1210/en.2015-2050.
19. Wepler, M.; Preuss, J. M.; Merz, T.; McCook, O.; Radermacher, P.; Tuckermann, J. P.; Vettorazzi, S. Impact of downstream effects of glucocorticoid receptor dysfunction on organ function in critical illness-associated systemic inflammation. Intensive Care Med. Exp. 2020, 8, 37, doi: 10.1186/s40635-020-00325-Z.
20. Ziv, L.; Muto, A.; Schoonheim, P. J.; Meijsing, S. H.; Strasser, D.; Ingraham, H. A.; Schaaf, M. J. M.; Yamamoto, K. R.; Baier, H. An affective disorder in zebrafish with mutation of the glucocorticoid receptor. Mol. Psychiatry 2013, 18, 681-691, doi: 10.1038/mp.2012.64.
21. Halima, M.; Ayed, K. Al; Kroos, S.; Kampstra, A. S. B.; Lelieveld, L. T.; Narayanan, A. Glycosylated glucocorticoid prodrugs show strongly reduced side effects due to specific Glucosylceramidase Beta 2-mediated activation in inflamed tissue Glucocorticoid receptor-dependent anti-inflammatory effects of glycosylated ginsenosides require deg.
22. van Pomeren, M.; Peijnenburg, W. J. G. M.; Brun, N. R.; Vijver, M. G. A Novel Experimental and Modelling Strategy for Nanoparticle Toxicity Testing Enabling the Use of Small Quantities. Int. J. Environ. Res. Public Health 2017, 14, doi: 10.3390/ijerph14111348.
23. Lelieveld, L. T.; Mirzaian, M.; Kuo, C.-L.; Artola, M.; Ferraz, M. J.; Peter, R. E. A.; Akiyama, H.; Greimel, P.; van den Berg, R. J. B. H. N.; Overkleeft, H. S.; et al. Role of beta-glucosidase 2 in aberrant glycosphingolipid metabolism: model of glucocerebrosidase deficiency in zebrafish. J. Lipid Res. 2019, 60, 1851-1867, doi: 10.1194/jlr.RA119000154.
24. Lelieveld, L. T. Zebrafish as research model to study Gaucher disease: Insights into molecular mechanisms; 2020.
25. Park H., Lee, M-K and Park, Q-H. Pattern of molecular aggregation of ginsenosides in aquous solution. J. Korean Agricultural Chemical society 1986, 29(2) 198-206
26. ZFIN The Zebrafish Information Network [Internet]. [cited 2020 Dec. 23]. Available from: https://zfin.org/10
27. Halima M, He M. Glycosylated ginsenosides exert their anti-inflammatory activities without promoting side effects by Gba2 mediated cleavage of glucose groups at the inflammation environment. 2020.

1. A ginsenoside formulation comprising a protopanaxatriol (PPT)-type ginsenoside at least partly incorporated in a micell, said micelle having a molecular weight of at least 10.000 Da.

2. The ginsenoside formulation according to claim 1, wherein said micelle has a molecular weight of at least 100.000 Da.

3. The ginsenoside formulation according to claim 1, wherein said ginsenoside is selected from Re, Rf, Rg1, Rg2, Rh1 or combinations thereof.

4. A pharmaceutical composition comprising a ginsenoside formulation according to claim 1 and a pharmaceutically acceptable carrier.

5. The pharmaceutical composition according to claim 4, further comprising a glucocorticoid (GC).

6. The pharmaceutical composition according to claim 5, wherein said GC is at least partly encapsulated in said micelle.

7. The pharmaceutical composition according to claim 4, wherein said pharmaceutical composition is formulated as a cream, a lotion, a balm, a hydrogel, an ointment, a foam, a gel, a spray, a tablet, a capsule, a lozenge, a topical solution, a topical suspension, an aerosol, an injectable or a syrup.

8. The pharmaceutical composition according to claim 5, wherein the molar ratio between said GC and said ginsenoside is between about 1:1 and about 1:2000.

9. (canceled)

10. The method according to claim 9, wherein said inflammatory disease is selected from asthma, allergic rhinitis, hay fever, urticaria, atopic eczema, chronic obstructive pulmonary disease, inflammation of the joints, muscles and tendons, lupus, inflammatory bowel disease, such as Crohn's disease and ulcerative colitis, giant cell arteritis and polymyalgia rheumatica, and multiple sclerosis.

11. The method according to claim 9, wherein said GC is administered in a therapeutically ineffective amount or wherein said ginsenoside is administered in a therapeutically ineffective amount, or both.

12. (canceled)

13. A method for preparing a ginsenoside formulation according to claim 1, comprising
(a) providing a ginsenoside extract from a plant or plant part of the genus Panax;
(b) contacting said ginsenoside extract with an aqueous solvent mixture to obtain a ginsenoside mixture;
(c) at least partly removing said aqueous solvent mixture from said ginsenoside mixture; and optionally
(d) reconstituting the ginsenoside mixture obtained in step c) in water to obtain a ginsenoside formulation.

14. The method according to claim 13, wherein said aqueous solvent mixture comprises a mixture of water and a non-aqueous solvent.

15. The method according to claim 13, comprising subjecting said ginsenoside extract to a step of column chromatography using a non-polar resin as a stationary phase or comprising subjecting said ginsenoside extract to a step of decolorization, or both.

16. (canceled)

17. (canceled)

18. A method of prevention or treatment of an inflammatory disease, comprising administering a GC and a PPT-type ginsenoside in a molar ratio of between about 1:1 and about 1:2000 to a subject in need thereof.

19. The method according to claim 18, wherein said GC is administered in a therapeutically ineffective amount.

20. The method according to claim 18, wherein said ginsenoside is selected from Re, Rf, Rg1, Rg2, Rh1 or combinations thereof.

21. The method according to claim 18, wherein said ginsenoside is at least partly incorporated into a micelle, said micelle having a molecular weight of at least 10.000 Da.

22. A method of preventing or treating an inflammatory disease, comprising administering a ginsenoside formulation according to claim 1 to a subject in need thereof.

23. The ginsenoside formulation according to claim 1, wherein said ginsenoside is Rg1, Re or a combination thereof.

24. The pharmaceutical composition according to claim 5, wherein said GC is dexamethasone, beclomethasone or a combination thereof.
